(19) United States
(12) Patent Application Publication
(10) Pub. No.: US 2025/0367023 A1
(43) Pub. Date: Dec. 4, 2025

(54) CRYO METHODS AND SYSTEMS FOR MEDICAL TREATMENTS

(71) Applicant: R2 Technologies, Inc., Dublin, CA (US)

(72) Inventors: Kevin SPRINGER, Livermore, CA (US); Gregory F. HIRTH, Pleasanton, CA (US); Timothy Dwaine HOLT, Miami, FL (US); Kristine TATSUTANI, Redwood City, CA (US); Nathan WONG, Oakland, CA (US)

(73) Assignee: R2 Technologies, Inc., Dublin, CA (US)

(21) Appl. No.: 19/300,475

(22) Filed: Aug. 14, 2025

Related U.S. Application Data
(63) Continuation of application No. PCT/US2024/019398, filed on Mar. 11, 2024.
(60) Provisional application No. 63/451,527, filed on Mar. 10, 2023.

(51) Int. Cl. A61F 7/00

(52) U.S. Cl. CPC A61F 7/0053; A61F 7/007; A61F 2007/0052; A61F 2007/0056; A61F 2007/0075; A61F 2007/0087

(57) ABSTRACT

The present describes cryo methods and systems for medical treatments. These medical treatments include treatments for a localized psoriatic plaque, treatments provided before, during, or after a medical procedure, and treatments to address skinflammation or an inflammatory condition. One aspect relates to a method of providing a static freeze treatment of a localized psoriatic plaque. The method includes exfoliating the psoriatic plaque and masking non-lesional tissue around and/or adjacent to the psoriatic plaque. The psoriatic plaque is divided into a plurality of treatment areas. Treatment is applied to the psoriatic plaque in each of the treatment areas via static application of cooling to the tissue in each treatment area.

### CROSS-REFERENCES TO RELATED APPLICATIONS

The present application is a Continuation of PCT/US2024/019398 filed Mar. 11, 2024, and entitled “CRYO METHODS AND SYSTEMS FOR MEDICAL TREATMENTS”, which claims the benefit of U.S. Provisional Application No. 63/451,527, filed on Mar. 10, 2023, and entitled “CRYO METHODS AND SYSTEMS FOR MEDICAL TREATMENTS”, the full disclosures which are incorporated herein by reference in their entirety for all purposes. This application is related to U.S. application Ser. No. 17/579,499, filed on Jan. 19, 2022, and entitled “DERMATOLOGICAL TREATMENTS AND SYSTEMS EMPLOYING COOLING, TOPICALS, AND/OR ABRASION”, the entirety of which is hereby incorporated by reference herein.

### BACKGROUND

Cryotherapy is the local or general use of cold in medical therapy. Cryotherapy can include the controlled freezing of biological tissue, which controlled freezing of biological tissue, such as skin tissue, can produce various effects. Certain tissue freezing procedures and devices, such as conventional cryoprobes, can cause severe freezing of tissue and generate cellular and visible skin damage.

Improved treatments, devices, and systems have been developed to improve the consistency of skin freezing. For example, it has been observed that moderate degrees of freezing (e.g., −4 to −30 degrees Celsius) at shorter time frames (e.g., 30 to 60 seconds) can produce particular dermatological effects. Cryotherapy can be provided using a variety of techniques including the direct application of a cryogen spray to the skin of the patient or the application of a cooled probe or plate to the skin of the patient. Exemplary methods and devices are described in: U.S. Patent Publication No. 2011/0313411, filed on Aug. 7, 2009, and entitled “METHOD AND APPARATUS FOR DERMATOLOGICAL HYPOPIGMENTATION”; U.S. Patent Publication No. 2014/0303696, filed on Nov. 16, 2012, and entitled “METHOD AND APPARATUS FOR CRYOGENIC TREATMENT OF SKIN TISSUE”; U.S. Patent Publication No. 2014/0303697, filed on Nov. 16, 2012, and entitled “METHOD AND APPARATUS FOR CRYOGENIC TREATMENT OF SKIN TISSUE”; U.S. Patent Publication No. 2015/0223975, filed on Feb. 12, 2015, and entitled “METHOD AND APPARATUS FOR AFFECTING PIGMENTATION OF TISSUE”; U.S. Patent Publication No. 2017/0065323, filed on Sep. 6, 2016, and entitled “MEDICAL SYSTEMS, METHODS, AND DEVICES FOR HYPOPIGMENTATION COOLING TREATMENTS”; U.S. Pat. No. 10,765,467, filed on Sep. 6, 2016, and entitled “Medical Systems, Methods, And Devices For Hypopigmentation Cooling Treatments”; U.S. Patent Publication No. 2017/0348143, filed on Jun. 2, 2017, and entitled “MEDICAL METHODS AND SYSTEMS FOR SKIN TREATMENT”, U.S. Publication No. 2019-0000524, filed on Jun. 27, 2018, and entitled “Dermatological Cryospray Devices Having Linear Array Of Nozzles And Methods Of Use”, U.S. Publication No. 2020-0214757, filed on Dec. 20, 2019, and entitled “Automated Dermatological Cryospray Treatment Planning System”, U.S. Publication No. 2020-0214757, filed on Dec. 20, 2019, and entitled “Automated Dermatological Cryospray Treatment Planning System”, U.S. Publication No. 2021-0407201, filed on Jun. 16, 2021, and entitled “Time-Of-Flight (Tof) Camera Systems And Methods For Automated Dermatological Cryospray Treatments”, the entirety of each of which is hereby incorporated by reference herein.

While the treatment of skin or a localized lesion can be accomplished with cryotherapy, it may be desirable to provide improved methods, systems, and devices for cryotherapy. In particular, improved designs, controls and parameters associated with cryogen delivery to achieve consistent and reliable skin freezing and/or cooling and desired skin treatment effect may be of benefit. Accordingly, improved dermatological treatments and systems are desirable.

### BRIEF SUMMARY

Embodiments disclosed herein relate to methods and systems for delivering treatments to a patient. These treatments can be provided to address a medical condition. This can range from treatment of a specific ailment such as psoriasis, moderate to severe psoriasis, acne, atopic dermatitis, eczema, and/or rosacea, to treatment of inflammation arising subsequent to a medical procedure, and/or to treatment of generalized inflammation such as may arise from exposure to the sun.

These treatments can be static freeze treatments or motion cooling treatments. The freeze treatment can be provided to patients with a localized lesion, such as a localized psoriatic plaque. The freeze treatment can include identifying a region to treat, applying a grid to that region, applying a coupling fluid to the region, and applying freezing to the region to be treated. This freeze treatment can be repeatedly provided to the patient. In some embodiments, the freeze treatment can be provided to the patient until the localized lesion has improved and/or healed.

The motion cooling treatment can be provided to patients to address specific skin disorders, subsequent to providing a medical procedure to the patient, and/or to address general inflammation. The motion cooling treatment can, in some embodiments, thermally modulate epidermal nerve endings to effect and/or mitigate neuroinflammation. In some embodiments, for example, the motion cooling treatment can be provided to facilitate healing and to decrease development of scar tissue such as, for example, development of a keloid scar and/or decrease occurrence of pigment alteration arising from healing.

The motion cooling treatment can be provided to patients with skin disorders affecting larger areas of skin, such as moderate to severe psoriasis, acne, atopic dermatitis, eczema, and/or rosacea. In some embodiments, different topicals can be used in connection with the motion cooling treatment to treat different skin conditions.

The motion cooling treatment can be provided to patients before, during, and/or after a medical procedure. In such an embodiment, the motion cooling treatment can be provided to facilitate healing and/or to treat or mitigate edema, redness, pain, injury including thermal injury, inflammation, and/or swelling. In some embodiments, different topicals can be used in connection with the motion cooling treatment to treat different ailments subsequent to the medical procedure.

The medical procedure can be a non-invasive or minimally invasive medical procedure such as, for example, a dermatologic procedure, a non-ablative, partially-ablative, or ablative laser procedure including for example a Fraxel® laser procedure, a microneedling procedure, an RF microneedling procedure, a radiofrequency procedure, a focused ultrasound treatment, an intense pulsed light or broadband light procedure, a powered facial procedure, an injection such as a botulinum toxin injection or filler injection, or the like. In the case of a non-invasive or minimally invasive medical procedure, the motion cooling treatment can be provided before, during, and/or immediately after completion of the non-invasive or minimally invasive medical procedure, or a short time after completion of the non-invasive or minimally invasive medical procedure. In some embodiments, for example, the motion cooling treatment can be provided within one hour of completion of the non-invasive or minimally invasive medical procedure. In some embodiments, the motion cooling treatment provided before the non-invasive or minimally invasive medical procedure can be provided within approximately 15 minutes of the start of the non-invasive or minimally invasive medical procedure.

The medical procedure can be a surgical procedure such as a procedure in which an incision is created in a patient. The surgical procedure can include, for example, a plastic surgery such as a facelift, a blepharoplasty, rhinoplasty, lip augmentation, otoplasty, genioplasty, cheek augmentation, jaw reduction, or the like. In some embodiments, the treatment is applied before the surgical procedure and/or after the incision has closed, which can be, one or several days after completion of the surgical procedure, such as, for example, one week after completion of the surgical procedure.

The motion cooling treatment can be provided to treat and/or to minimize low-level inflammation, for example, skinflammation, such as can arise from sun exposure and/or sun damage to the skin. In some embodiments, this low-level inflammation can be persistent over a long period of time, and may lead to, for example, premature aging including, premature development of wrinkling, skin thinning, discoloration such as age spots, or the like.

The motion cooling treatments can be provided once, or multiple times over a period of one or several months until the patient's condition improves. One aspect disclosed herein relates to a method of providing a static freeze treatment of a localized psoriatic plaque. The method includes exfoliating the psoriatic plaque, dividing the psoriatic plaque into a plurality of treatment areas, and applying treatment to the psoriatic plaque in each of the treatment areas via static application of a cooling to the tissue in each treatment area.

In some embodiments, cooling is applied to tissue in each treatment area via spraying of cryogen onto tissue in each treatment area. In some embodiments, spraying cryogen onto tissue in each treatment area includes robotic controlled spraying of cryogen onto tissue in each treatment area. In some embodiments, cooling is applied to tissue in each treatment area via application of a cold plate to tissue in each treatment area.

In some embodiments, each of the plurality of treatment areas has the same size. In some embodiments, dividing the psoriatic plaque into a plurality of treatment areas includes applying a grid to the psoriatic plaque via a template. In some embodiments, the template can be a flexible planar member defining a plurality of equally spaced holes through which the skin of and/or around the psoriatic plaque can be marked to apply the grid to the psoriatic plaque. In some embodiments, the holes of the template have an equal spacing that is less than a dimension of the cold plate. In some embodiments, the grid can be applied to the skin as, for example, a tattoo pattern. In some embodiments, the grid can be applied to the skin via, for example, a temporary tattoo.

In some embodiments, applying the treatment to the psoriatic plaque includes applying cooling to each grid of the psoriatic plaque and holding the cooling at that location for a predetermined period of time. In some embodiments, the cooling can be applied via a cold plate, and some embodiments, the cooling can be applied via direct application of a cryogen to skin in each grid. In some embodiments, the cryogen can be applied to the skin in each grid via, for example, spraying cryogen onto the skin. In some embodiments, the cryogen can be robotically sprayed onto the skin.

In some embodiments, the predetermined period of time is between approximately 10 seconds and approximately 30 seconds, and/or is between approximately 11 seconds and approximately 26 seconds. In some embodiments, the cold plate has a temperature of between approximately −5° C. and −30° C., between approximately −10° C. and −20° C., between approximately −15° C. and −20° C., and/or of approximately −17° C. during application of the treatment. In some embodiments, the skin can be cooled to a temperature below 0° C., and can be held at this temperature for a period of between approximately 11 seconds and approximately 30 seconds. In some embodiments, the treatment includes a cooling period, and a re-warming period.

In some embodiments, the method includes applying a coupling fluid, which can be a gel to the psoriatic plaque before applying the treatment to the psoriatic plaque via the cold plate. In some embodiments, the coupling fluid has a viscosity of between approximately 10 centipoise and approximately 1500 centipoise, between approximately 20 centipoise and approximately 1000 centipoise, between approximately 50 centipoise and approximately 600 centipoise, between approximately 600 centipoise and approximately 1000 centipoise, and/or of between approximately 200 centipoise and approximately 400 centipoise. In some embodiments, the coupling fluid is approximately 98% water and approximately 2% thickening agent. In some embodiments, the coupling fluid has a freezing point higher than the temperature of the cold plate. In some embodiments, the coupling fluid can be configured to hydrate the tissue being treated to thereby facilitate heat transfer out of the treated tissue and/or to facilitate ice propagation into the treated tissue to thereby facilitate freezing of that tissue.

In some embodiments, the exfoliation can include at least one of mechanical exfoliation or chemical exfoliation. In some embodiments, the exfoliation can include application of an adhesive material, and removal of the adhesive material. In some embodiments, removal of the adhesive material removes loose portions of the psoriatic plaque. In some embodiments, the exfoliation can include exfoliation via an abrading device.

In some embodiments, the method includes washing the exfoliated psoriatic plaque to remove loose debris. In some embodiments, the exfoliated psoriatic plaque can be washed with at least one of alcohol such as, for example, isopropyl alcohol, water, or a cleanser.

In some embodiments, the method includes masking via application of a waterproof, and/or thermally insulating material to non-lesional skin adjacent to the psoriatic plaque. In some embodiments, the waterproof material includes at least one of waterproof tape, and an elastomeric material. In some embodiments, the elastomeric material is sprayed on the skin and/or is brushed on the skin.

In some embodiments, the static freeze treatment can be repeatedly provided to the psoriatic plaque. In some embodiments, the static freeze treatment can be provided once a week, every other week, once a month, or at any other or intermediate interval.

One aspect disclosed herein relates to a method of providing a motion treatment of at least one diffuse psoriatic plaque. The method can include, in some embodiments, exfoliating the diffuse psoriatic plaque. The method can include dividing the diffuse psoriatic plaque into a plurality of treatment segments. The method can include applying a coupling fluid to the diffuse psoriatic plaque. The method can include applying treatment to the diffuse psoriatic plaque in each of the treatment segments via movingly applying cooling across a surface of tissue in each segment for a predetermined time period.

In some embodiments, movingly applying cooling across the surface of tissue in each segment includes movingly spraying cryogen across the surface of tissue in each segment. In some embodiments, spraying cryogen across the surface of tissue in each segment includes robotic controlled spraying of cryogen across the surface of tissue in each segment. In some embodiments, movingly applying cooling across the surface of tissue in each segment includes rubbing a cold plate across the surface of tissue in each segment.

In some embodiments, the predetermined time period is approximately 10 minutes per segment. In some embodiments, the cold plate has a temperature of between approximately −16° C. and approximately 4° C., and/or between approximately −6° C. and approximately 1° C.

In some embodiments, the method includes applying a coupling fluid, which can be a gel, to a region of skin affected by an inflammation-related skin condition such as, for example, psoriasis, atopic dermatitis, eczema, acne, or rosacea before applying the treatment to the affected skin via the cold plate. In some embodiments, the coupling fluid has a viscosity and lubricity to facilitate moving the cold plate across the surface of the tissue. In some embodiments, the coupling fluid has a freezing point lower than the temperature of the cold plate.

In some embodiments, the method can include exfoliating the psoriatic plaque. In some embodiments, the exfoliation can comprise at least one of mechanical exfoliation, or chemical exfoliation. In some embodiments, the exfoliation can include application of an adhesive material, and removal of the adhesive material. In some embodiments, removal of the adhesive material removes loose portions of the psoriatic plaque. In some embodiments, the exfoliation can include exfoliation via an abrading device. In some embodiments, the method includes washing the exfoliated psoriatic plaque to remove loose debris. In some embodiments, the exfoliated psoriatic plaque can be washed with at least one of rubbing alcohol, water, or a cleanser.

In some embodiments, the method includes coupling a treatment tip to the cold plate. In some embodiments, applying treatment to the diffuse psoriatic plaque includes contacting the treatment tip to the surface of the tissue in each segment and moving the treatment tip coupled to the cold plate across the surface of tissue in each segment for the predetermined time period. In some embodiments, the treatment tip comprises a smooth tip.

One aspect disclosed herein relates to a method of providing a motion treatment. The method includes providing a medical procedure to a procedure area of skin of a patient, and identifying an area for receiving the motion treatment. In some embodiments, the area for receiving the motion treatment includes the procedure area. The method can include dividing the area for receiving the motion treatment into a plurality of treatment segments. The method can include applying a coupling fluid to the area for receiving the motion treatment. The method can include applying treatment to the area for receiving the motion treatment in each of the treatment segments via movingly applying cooling across a surface of tissue in each segment for a predetermined time period.

In some embodiments, the medical procedure can be a non-invasive or minimally invasive medical procedure. The non-invasive or minimally invasive procedure can include at least one of a non-ablative, partially-ablative, or ablative laser procedure, a microneedling procedure, an RF microneedling procedure, a radiofrequency procedure, a focused ultrasound treatment, an intense pulsed light or broadband light procedure, a powered facial procedure, an injection such as a botulinum toxin injection or a filler injection, or the like. In some embodiments, the treatment is applied to the area for receiving the motion treatment within one hour of providing the non-invasive or minimally invasive medical procedure.

In some embodiments, the medical procedure can be a surgical procedure. In some embodiments, providing the surgical procedure creates an incision. In some embodiments, the treatment is applied after the incision has closed.

In some embodiments, movingly applying cooling across the surface of tissue in each segment includes movingly spraying cryogen across the surface of tissue in each segment. In some embodiments, spraying cryogen across the surface of tissue in each segment includes robotic controlled spraying of cryogen across the surface of tissue in each segment. In some embodiments, movingly applying cooling across the surface of tissue in each segment includes rubbing a cold plate across the surface of tissue in each segment.

In some embodiments, the treatment can be applied to the area for receiving the motion treatment before providing the medical procedure. In some embodiments, the treatment can be applied to the area for receiving the motion treatment during the providing of the medical procedure. In some embodiments, the treatment can be applied to the area for receiving the motion treatment after providing the medical procedure.

One aspect of the present disclosure relates to a method of providing a motion treatment to at least a portion of skin affected by an inflammatory condition. The method can include dividing the portion of skin into a plurality of treatment segments. The method can include applying a coupling fluid to the portion of skin. The method can include applying treatment to the portion of skin in each of the treatment segments via movingly applying cooling across a surface of tissue in each segment for a predetermined time period.

In some embodiments, the inflammatory condition can include at least one of psoriasis, atopic dermatitis, eczema, acne, or rosacea.

In some embodiments, movingly applying cooling across the surface of tissue in each segment includes movingly spraying cryogen across the surface of tissue in each segment. In some embodiments, spraying cryogen across the surface of tissue in each segment includes robotic controlled spraying of cryogen across the surface of tissue in each segment. In some embodiments, movingly applying cooling across the surface of tissue in each segment comprises moving a cold plate across the surface of tissue in each segment. In some embodiments, the predetermined time period is approximately 10 minutes per segment.

In some embodiments, the cold plate has a temperature of between approximately −16° C. and approximately 1° C. In some embodiments, the coupling fluid has a viscosity and lubricity to facilitate moving the cold plate across the surface of the tissue. In some embodiments, the coupling fluid has a freezing point lower than a temperature of the cold plate.

In some embodiments, the exfoliation can include at least one of mechanical exfoliation; or chemical exfoliation. In some embodiments, the exfoliation can include application of an adhesive material and removal of the adhesive material. In some embodiments, removal of the adhesive material removes loose portions of the psoriatic plaque. In some embodiments, the exfoliation can include exfoliation via an abrading device.

In some embodiment, the method can include washing the exfoliated psoriatic plaque to remove loose debris. In some embodiments, the exfoliated psoriatic plaque can be washed with at least one of rubbing alcohol, water, or a cleanser.

In some embodiments, the method can include coupling a treatment tip to the cold plate. In some embodiments, applying treatment to the portion of skin includes contacting the treatment tip to the surface of the tissue in each segment and moving the treatment tip coupled to the cold plate across the surface of tissue in each segment for the predetermined time period. In some embodiments, the treatment tip can be a smooth tip.

One aspect relates to a method of providing a motion treatment. The method includes providing a medical procedure to a procedure area of skin of a patient, identifying an area for receiving the motion treatment, the area including the procedure area, applying a treatment topical to the area for receiving the motion treatment, and applying treatment to the area for receiving the motion treatment with a cooling treatment system. The cooling treatment system includes a body including a chiller, a handpiece including a cold plate and a cooler, and a cable connecting the handpiece and the body. In some embodiments, the cooler is thermally coupled to the cold plate and can control a temperature of the cold plate. In some embodiments, a coolant can circulate between the chiller and the cooler via the cable.

In some embodiments, the medical procedure can be a non-invasive or minimally invasive medical procedure. In some embodiments, the non-invasive or minimally invasive medical procedure includes at least one of a laser procedure, a microneedling procedure, an RF microneedling procedure, a radiofrequency procedure, a focused ultrasound treatment, an intense pulsed light or broadband light procedure, or a powered facial procedure. In some embodiments, the treatment is applied to the area for receiving the motion treatment within one hour of providing the non-invasive or minimally invasive medical procedure.

In some embodiments, the medical procedure can be a surgical procedure. In some embodiments, providing the surgical procedure creates an incision. In some embodiments, the treatment is applied after the incision has closed.

In some embodiments, the method includes coupling a treatment tip to the cold plate. In some embodiments, applying treatment to the area for receiving the motion cooling treatment includes contacting the treatment tip to the surface of tissue in the area and moving the treatment tip coupled to the cold plate across the surface of tissue in the area for a predetermined time period.

In some embodiments, the predetermined time period is approximately 10 minutes per segment. In some embodiments, the cold plate has a temperature of between approximately −16° C. and approximately 4° C. In some embodiments, the treatment tip can be a smooth tip.

In some embodiments, the cold plate includes a distal protrusion, a camming surface extending proximally and radially away from a proximal end of the distal protrusion, and a retention depression connecting to a proximal end of the camming surface. In some embodiments, the cold plate further includes a plurality of axial grooves located around a perimeter of a junction of the proximal end of the camming surface and the retention depression. In some embodiments, the retention depression extends to a retention depth below the proximal end of the camming surface. In some embodiments, the axial grooves have a groove depth equal to the retention depth.

In some embodiments, the tip includes an exterior housing defining a receptacle, and a retention feature located inside the receptacle. In some embodiments, the receptacle can receive at least portions of the cold plate. In some embodiments, the retention feature can engage with the retention depression of the cold plate to couple the tip to the cold plate.

In some embodiments, the method includes applying coupling fluid to at least one of the cold plate and the receptacle of the tip, and coupling the tip to the cold plate. In some embodiments, coupling the tip to the cold plate expels at least a portion of the coupling fluid via at least one of the plurality of axial grooves. In some embodiments, the coupling fluid is different than the treatment topical. In some embodiments, the coupling fluid has a freezing point lower than a temperature of the cold plate.

In some embodiments, the cooler can be a thermoelectric cooler (TEC). In some embodiments, applying the treatment to the area for receiving the motion treatment with a cooling treatment system includes controlling a temperature of the cold plate with the cooler.

### BRIEF DESCRIPTION OF THE DRAWINGS

FIG. 1 is a schematic illustration of one embodiment of a cooling treatment system.

FIG. 2 is a schematic illustration of electrical components of the cooling treatment system.

FIG. 3 is a perspective view of one embodiment of the handpiece.

FIG. 4 is a front view of two handpieces, each having a different cold plate.

FIG. 5 is a perspective view of one embodiment of a cold plate.

FIG. 6 is a side view of one embodiment of a cold plate

FIG. 7 is a perspective view of one embodiment of a smooth tip.

FIG. 8 is a side view of one embodiment of a smooth tip.

FIG. 9 is a section view of one embodiment of a smooth tip.

FIG. 10 is a perspective view of one embodiment of a textured tip.

FIG. 11 is a side view of one embodiment of a textured tip.

FIG. 12 is a section view of one embodiment of a textured tip.

FIG. 13 is a flowchart illustrating one embodiment of a process for delivering a static freeze treatment.

FIG. 14 is a depiction of a localized lesion.

FIG. 15 is a depiction of a patient's knee including a masked lesion divided by a grid.

FIG. 16 is a front view of a template.

FIG. 17 depicts a first patient's knee after application of the static freeze treatment to a selected one of the treatment areas.

FIG. 18 depicts a second patient's knee after application of the static freeze treatment to a selected one of the treatment areas.

FIG. 19 is a plurality of images of two lesions on a patient showing treatment efficacy over time.

FIG. 20 is a flowchart illustrating one embodiment of a process for providing a motion cooling treatment.

FIG. 21 is a flowchart illustrating another embodiment of a process for providing a motion cooling treatment.

FIG. 22 is a depiction of a diffuse lesion on a leg of a patient.

### DETAILED DESCRIPTION

Cooling based treatments are frequently used to address a wide range of health and aesthetic issues. These issues can include, for example, the ablation of benign lesions such as, for example, acne—vulgaris, cystic; acne keloidalis; adenoma sebaceum; alopecia areatea; angiokeratomas; angiokeratoma of Fordyce; atypical fibroxanthoma; cherry angiomas; chonrodermatitis nodularis helicis; chromoblastomycosis; clear cell acanthoma; condyloma acuminatum; dermatofibroma; disseminated superficial actinic porokeratosis; elastosis perforans serpiginosa; epidermal nevus; erosive adenomatosis of the nipple; folliculitis keloidalis; granuloma annulare; granuloma faciale; granulomaa pyogenicum; hemangioma; herpes labialis; idiopathic guttate hypomelanosis; Kyrle's disease; leishmaniasis; lentigines; lentigo simplex; lichen sclerosus et atrophicus of vulva; lupus erythematosus; lymphangioma; lymphocytoma cutis; molluscum contagiosum; mucocele; myxoid cyst; orf; porokeratosis plantaris discreta; porokeratosis of Mibelli; prurigo nodularis; pruritus ani; psoriasis; rhinophyma; rosacea; sarcoid; sebaceous hyperplasia; seborrheic keratosis; solar lentigo; syringoma; trichiasis; trichoepithelioma; varicose veins; venous lakes; verrucae—periungual, plane, vulgaris, filiform, plantar; xanthoma; acne scar; keloids; cutaneous horn; hypertrophic scar; ingrown toenail; skin tags; tattoos; freckles; spider naevus; capillary haemangioma; cavernous haemangioma; milia; trichilemmal cyst; steatocystoma multiplex; hidrocystoma; acrokeratosis veruciformis; dermatosis papulose nigra; hyperkeratosis naevoid of nipple; benign lichenoid keratosis; angiofibromas; and angiomas. In some embodiments, cooling based treatments can be used to treat pre-malignant skin conditions such as, for example: actinic keratosis; leukoplakia; Bowen disease; erythroplasia of Quyrat; keratoacanthoma; and lentigo maligna, and can be used to treat malignant skin conditions such as, for example: basal cell carcinoma; Kaposi sarcoma; squamous cell carcinoma; and melanoma.

Some of these treatments have been specifically designed to cause skin healing, achieve a desired cosmetic benefit, and/or to change a color of the skin via the creation of skin lightening or of skin darkening. This can include minimizing lines and/or wrinkles, improving skin roughness, improving evenness of skin tone, or the like. While such treatments have been long used, their previous effectiveness has been limited.

Some of these health and/or aesthetic issues are tied to inflammation in that addressing the underlying inflammation can mitigate the manifestation of the health and/or aesthetic issue.

Some such conditions include, for example, psoriasis, acne, atopic dermatitis, eczema, and rosacea. By applying either a freezing or a cooling treatment, these conditions can be improved, and their visual and/or symptomatic manifestation can be minimized.

The present disclosure relates to systems, devices, and methods of treating portions of a patient's skin. This can include treating of local or diffuse lesions, such as local psoriatic plaques (psoriasis plaques) or diffuse psoriatic plaques. This can further include treating, for example, rosacea, eczema, atopic dermatitis, and acne.

Such treatments can include either a static treatment, or a dynamic treatment. A static treatment, also referred to herein as a static freeze treatment, can be used in treating a smaller area, such as occurs when treating a localized psoriatic plaque, and a dynamic treatment, also referred to herein as a motion treatment or as a cooling motion treatment, can be performed when treating diffuse conditions such as diffuse psoriatic plaques, atopic dermatitis, eczema, rosacea, and/or acne. In some embodiments, these treatments, whether static or dynamic can treat inflammation related conditions. In some embodiments, these treatments, whether static or dynamic can be utilized to facilitate healing and/or to mitigate risk of adverse effects after completion of a medical procedure such as, for example, after completion of non-invasive or minimally invasive procedure such as, for example, a non-ablative, partially-ablative, or ablative laser procedure including for example a Fraxel® laser procedure, a microneedling procedure, an RF microneedling procedure, a radiofrequency procedure, a focused ultrasound treatment, an intense pulsed light or broadband light procedure, a powered facial procedure an injection such as a botulinum toxin injection or filler injection, or the like. In some embodiments, these motion cooling treatments can be provided before, during, and/or after the non-invasive or minimally invasive medical procedure to facilitate healing, and/or to mitigate the risk of adverse effects from the procedure. In some embodiments, the motion cooling treatment can be provided before and/or during the medical procedure to suppress pain and/or increase user comfort during the procedure.

In some embodiments, these treatments, whether static or dynamic can be utilized to facilitate healing and/or to mitigate risk of adverse effects after completion of a medical procedure such as a surgical procedure. In some embodiments, for example, these treatments can be provided before and/or after surgery to facilitate healing and to decrease development of scar tissue such as, for example, development of a keloid scar and/or decrease occurrence of pigment alteration arising from healing.

A static treatment can include preparing a location on the patient's body and then holding a cold plate at that location for a predetermined period of time. The preparing of the location to be treated can include exfoliating the region to be treated. In the event that the region to be treated includes and/or is a psoriatic plaque, this can include exfoliating the plaque. In some embodiments, exfoliating the plaque can improve the efficacy of the treatment by facilitating cooling of tissue covered by the plaque, or in other words, underlying the plaque. Specifically, in some embodiments, the all or portions of the plaque can function as insulation to inhibit cooling of the tissue underneath the plaque. By exfoliating the plaque, this insulative quality can be diminished, thereby improving the cooling of the tissue beneath the plaque.

The exfoliation can use different techniques to remove tissue, such as dead and/or loose tissue from the plaque, including mechanical exfoliation or a chemical exfoliation. For example, this exfoliation can be performed via an abrading device, or via applying and removing an adhesive material to the plaque. For example, tape, such as medical bandaging tape can be applied to and removed from the plaque, thereby removing loose material from the plaque which loose material adheres to the tape. Upon completion of the exfoliation, the area to be treated, and specifically, the exfoliated area can be washed and/or cleansed to remove any debris left from the exfoliation. This washing and/or cleansing can be performed with water, alcohol, a cleanser, or the like.

The area to be treated can divided into a plurality of treatment areas. These areas can have a variety of shapes and sizes, which in some embodiments, can include one or several rectangles and/or squares that are sized to be approximately the same size as a cold plate used in providing the treatment. In some embodiments, each of the treatment areas can be a square or rectangle that is slightly smaller than the cold plate used in providing the treatment. For example, the cold plate can comprise a square having a length and wide of, for example, between approximately 5 mm and approximately 50 mm, between approximately 10 mm and approximately 40 mm, between approximately 20 mm and 30 mm, of approximately 7 mm, of approximately 25 mm, or any other or intermediate dimension. In some embodiments, the cold plate can comprise a circle having a diameter of between approximately 5 mm and approximately 50 mm, between approximately 10 mm and approximately 40 mm, between approximately 20 mm and 30 mm, of approximately 7 mm, of approximately 25 mm, or any other or intermediate diameter. As used herein “approximately” defines a range of +/−10% around the based number modified by “approximately.”

In some embodiments, for example, the treatment areas can have the same dimensions as the cold plate, and in some embodiments, the treatment areas can be slightly smaller than the cold plate. In some embodiments, sizing the treatment areas smaller than the cold plate can mitigate the risk of missing treatment of portions of the treatment areas, and specifically of missing treatment of portions around the periphery of the treatment areas. In some embodiments, for example, sizing the cold plate slightly larger than the treatment areas can prevent missing treatment portions around the periphery of the treatment areas by assuring that all treatment areas are contiguous and/or slightly overlapping.

For example, in some embodiments some or all of the treatment areas can have the same shape as the cold plate and have one or more dimensions that are smaller than the one or more dimensions of the cold plate. For example, the dimensions of the treatment areas can be between approximately 0 percent and approximately 25 percent smaller than the corresponding dimensions of the cold plate. For example, the cold plate can define a square having a length and width of approximately 25 mm, and the treatment areas can each comprise a square having a length and width of between approximately 20 mm and approximately 25 mm, and specifically having a length and width of approximately 23 mm.

The area to be treated can be divided into a plurality of treatment areas via application of a grid to the area to be treated, which grid defines the plurality of treatment areas. In some embodiments, a template can be used to apply the grid to the area to be treated. This template can comprise, for example, a flexible, planar member that can define a plurality of holes. In some embodiments, the template can be sufficiently flexible as to bend around contours of the patient's anatomy such that the grid can be applied to a non-planar surface on the patient's body.

In some embodiments, the template can include any desired number of holes. These holes, can in some embodiments, comprise equally spaced and/or non-equally spaced holes. In some embodiment, a template can include between 10 and 100 equally spaced holes, between 25 and 75 equally spaced holes, approximately 49 equally spaced holes, or any other or intermediate number of equally spaced holes. In some embodiments, applying the grid to the area to be treated can include positioning the template on the plaque, and marking the patient's skin through the holes in the template.

In some embodiments, the grid can be applied to the skin as, for example, a tattoo pattern. In some embodiments, this tattoo pattern can comprise a lined grid applied directly onto the skin. In some embodiments, this tattoo pattern can be temporarily applied to the skin. In some embodiments, this tattoo pattern can be temporarily applied to the skin via, for example, a temporary tattoo. In some embodiments the temporary tattoo can comprise a plurality of lines arranged in a grid and a plurality of holes between the lines, and specifically in the spaces defined by the grid. In some embodiments, these holes can assist in proper placement of the temporary tattoo over and on the lesion. In some embodiments, these holes can extend through the backing material of the temporary tattoo, and in some embodiments, can increase the flexibility of the temporary tattoo such that the temporary tattoo better conforms to the contours of the area being treated.

The preparing of the location to be treated can further include masking areas of non-lesional and/or healthy skin adjacent and/or close to the plaque. In some embodiments, this masking can prevent freezing and/or creation of a cold injury to the masked tissue. The masking can, in some embodiments, be performed before or after application of the grid to the patient's skin.

In some embodiments, tissue can be masked by application of some material to the skin. This material can be a waterproof and/or insulating material and can include, for example, waterproof tape. In some embodiments, waterproof tape can be applied, or more specifically, adhered to healthy skin surrounding the lesion to be treated. In some embodiments, the non-lesional and/or healthy skin can be masked by application of a waterproof, thermally insulating material, such as elastomer, which can be applied to the patient's skin via brush and/or spraying.

The preparation of the location to be treated can further include application of coupling fluid or water. The coupling fluid can be applied to the area to be treated. The coupling fluid can, in some embodiments, hydrate the tissue of the area to be treated and facilitate the propagation of ice crystals through the area to be treated and into the underlying tissue. In some embodiments in which the area to be treated comprises a localized plaque, the coupling fluid can be applied to the localized plaque and can hydrate the localized plaque and facilitate in the propagation of ice crystals into and through the plaque and into the skin underneath the plaque.

In some embodiments, the coupling fluid can fill in air pockets and gaps in the area to be treated. Specifically, in some embodiments, the coupling fluid can fill air pockets and/or gaps in the lesion skin (e.g., resulting air pockets after the removal of debris and dead cells during the exfoliation step) and helps promote consistent and effective freeze of the plaque.

In some embodiments, the coupling fluid can be, for example, a water-based coupling fluid such as a water-based gel. The water-based coupling fluid can, for example, comprise water and a thickening agent. In some embodiments, the coupling fluid can comprise between approximately 90% and approximately 99% water by weight, and/or can comprise approximately 98% water by weight. In some embodiments, the coupling fluid can comprise between approximately 10% and approximately 1% thickening agent by weight, and/or can comprise approximately 2% thickening agent by weight.

The coupling fluid can be configured to have a viscosity sufficient to allow the coupling fluid to penetrate into the plaque without running off of the plaque. However, the coupling fluid is also configurated with a viscosity not so high as to preclude or diminish its penetration into voids in the plaques. In some cases, the coupling fluid may also include a surfactant to reduce the surface tension of the coupling fluid and promote wetting of the tissue.

In some embodiments, this can be a viscosity of, for example, between approximately 10 centipoise and approximately 1500 centipoise, between approximately 20 centipoise and approximately 1000 centipoise, between approximately 50 centipoise and approximately 600 centipoise, between approximately 600 centipoise and approximately 1000 centipoise, and/or of between approximately 200 centipoise and approximately 400 centipoise.

Upon preparation of the area to be treated, the static treatment, which can be a freeze treatment, can be applied by cooling the cold plate to a desired temperature. In some embodiments, a freeze treatment can be a treatment that results in freezing of a water component of the skin in the treatment location. This freezing of the water component of the skin in the treatment location can include crystallization of the water component of the tissue in the treatment location. In some embodiments, the cold plate can be cooled to a desired temperature that can be, for example, below approximately −10° C., below approximately −15° C., between −10° C. and −25° C., to be approximately −17° C., or any other or intermediate temperature.

The cold plate can be applied to skin in one of the treatment areas, and can be held in contact with the skin in the one of the treatment areas for a predetermined period of time. For example, the cold plate can be held in static contact with skin in the one of the treatment areas for a time of between approximately 5 seconds and approximately 45 seconds, of between approximately 10 seconds and approximately 30 seconds, and/or of between approximately 11 seconds and approximately 26 seconds, or any other or intermediate amount of time. In some embodiments, the amount of time that the cold plate is held in contact with the skin of the one of the treatment areas varies based on a thickness or level of scale of the lesion, and specifically of the plaque being treated. In some embodiments, for example, the cold plate is held in contact with the skin of the one of the treatment areas for a relatively longer period of time when the scale and/or thickness of the lesion is relatively thicker, and in some embodiments, the cold plate is held in contact with the skin of the one of the treatment areas for a relatively shorter period of time when the scale and/or thickness of the lesion is relatively thinner.

In some embodiments, and while the cold plate is held against the patient's skin on one of the treatment areas, the temperature of the cold plate can be controlled according to one or several temperature profiles, also referred to herein as one or several treatment profiles, which temperature profiles can be predetermined. In some embodiments, this treatment profile can include a first period which can be a cooling period and a second period which can be a rewarming period. The cooling period can last, in some embodiments, between 11 and 30 seconds, and the rewarming period can last, in some embodiments, between approximately 5 seconds and approximately 15 seconds, and in some embodiments, can last approximately 6 seconds. In some embodiments, and during the first period, the temperature of the cold plate can be controlled to cause the skin at the treatment location to freeze, and during the second period, the temperature of the cold plate can be controlled to cause the skin at the treatment location to be rewarmed. In some embodiments, the cold plate can be controlled to a fixed temperature, to a stepped temperature, to a ramped temperature, or to any other desired temperature profile. In some embodiments, the rewarming period can thaw the frozen skin and/or can rewarm the frozen skin. In some embodiments, the skin can be rewarmed sufficient to thaw frozen tissue and/or such that the cold plate can be safely removed from the skin. In some embodiments, this can include rewarming the skin such that no portion of skin is frozen to the cold plate and/or until the cold plate has a temperature of above 0° C. and/or a temperature above 0° C. for a predetermined time period. The temperature of the cold plate can be controlled during the first period to be below approximately −10° C., below approximately −15° C., between −10° C. and −25° C., to be approximately −17° C., or any other or intermediate temperature. This can be repeated for each treatment area, thereby providing a freeze treatment to each of the treatment areas.

A cooling motion treatment can include preparing a location and then moving the cold plate and/or a tip which can be attached to the cold plate across the location for a duration of time to cool the tissue at that location. Like the static treatment, the preparing of the location to be treated can include exfoliating the region to be treated as described above. As described, this exfoliation removes tissue, such as dead and/or loose tissue from tissue, including diffuse plaque being treated, and thereby improving thermal conduction through the tissue. After exfoliation, the area to be treated, and specifically, the exfoliated area can be washed and/or cleansed to remove any debris left from the exfoliation. This washing and/or cleansing can be performed with water, alcohol, a cleanser, or the like.

The area to be treated by the cooling motion treatment can divided into a plurality of treatment segments. These segments can have a variety of shapes and sizes, and can be significantly larger than the dimensions of the cold plate to allow the cold plate to be moved across the skin of the treatment area during the treatment.

The preparation of the location to be treated for the cooling motion treatment can further include application of coupling fluid to the area to be treated. The coupling fluid can, in some embodiments, hydrate the tissue of the area to be treated and facilitate cooling of the area to be treated. In some embodiments, the coupling fluid can fill in air pockets and gaps in the area to be treated. Specifically, in some embodiments, the coupling fluid can fill air pockets and/or gaps in the lesion skin (e.g., resulting air pockets after the removal of debris and dead cells during the exfoliation step) and helps promote consistent and effective cooling of the area to be treated.

In some embodiments, the coupling fluid can be a water-based coupling fluid that can comprise, for example, comprise water and a thickening agent. In some embodiments, the coupling fluid can comprise between approximately 90% and approximately 99% water, and/or can comprise approximately 98% water. In some embodiments, the coupling fluid can comprise between approximately 10% and approximately 1% thickening agent, and/or can comprise approximately 2% thickening agent.

The coupling fluid can be configured to have a viscosity and a lubricity to facilitate providing of the cooling motion treatment. For example, the coupling fluid can have a viscosity sufficient to allow the coupling fluid to penetrate into the plaque without running off of the plaque. In some embodiments, this can be a viscosity of between, for example, approximately 200 centipoise and approximately 400 centipoise.

In some embodiments, the coupling fluid can be configured to have a freezing point or a phase transition temperature that is lower than the temperature of the cold plate during the cooling motion treatment. In some embodiments, for example, the cold plate can be cooled to a temperature of between approximately −16° C. and 4° C., and/or between approximately −6° C. and approximately 1° C. Thus, in some embodiments, the coupling fluid can have a freezing point or a phase transition temperature that is lower than, for example, approximately −16° C., lower than approximately −6° C. and/or lower than a temperature between approximately −16° C. and approximately 4° C., and/or between approximately −6° C. and approximately 1° C. In some embodiments, the freezing point of the coupling fluid can be between approximately 0° C. and approximately −40° C., between approximately −10° C. and-30° C., approximately −22° C., or any other or intermediate freezing point.

Upon preparation of the area to be treated, the motion cooling treatment can be applied by cooling the cold plate to a desired temperature and rubbing the cold plate across the surface of the skin in a treatment segment to cool that skin. This can be repeated until the motion cooling treatment has been applied to each of the treatment segments. In some embodiments, and during the motion cooling treatment, the cold plate can be cooled to a temperature of between approximately −16° C. and approximately 1° C., and the rubbing of the cold plate across the surface of the skin of a treatment segment can cool the skin in a treatment segment to a temperature of between approximately 0° C. and 10° C., between approximately 1° C. and 6° C., between approximately 2° C. and 4° C., approximately 2° C., or any other or intermediate temperature while the cooling motion treatment is applied to that treatment segment. In some embodiments, the cold plate can be rubbed across the treatment segment for a predetermined time period, which time period can vary based on the size of the treatment segment and/or the thickness of lesion and/or plaque in the treatment segment. In some embodiments, the cold plate can be rubbed across the surface of skin in the treatment segment for between approximately 2 minutes and approximately 25 minutes, for between approximately 5 minutes and approximately 20 minutes, for approximately 10 minutes, or for any other or intermediate amount of time.

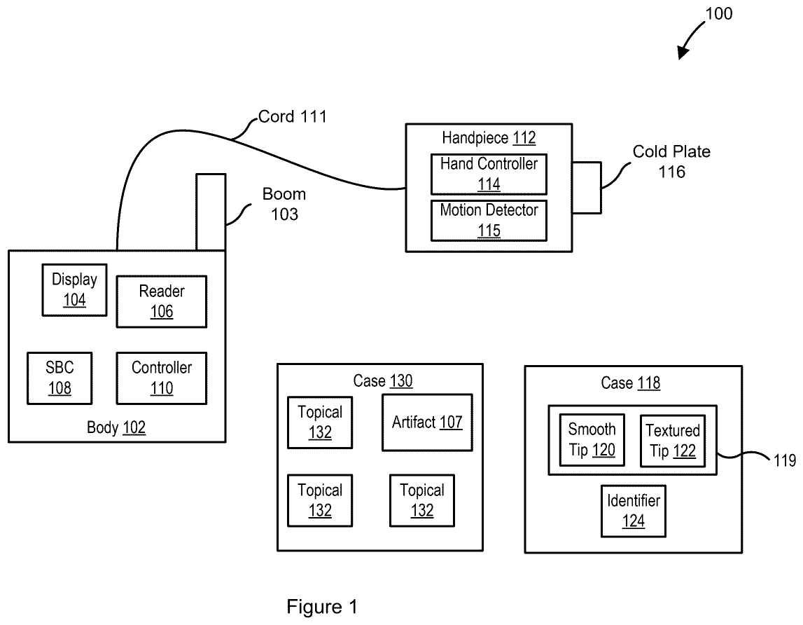

With reference now to FIG. 1, a schematic illustration of one embodiment of a cooling treatment system 100 is shown. Further details of the cooling treatment system 100 are found in U.S. Publication No. 2022/0226149, filed on Jan. 19, 2022, and entitled “DERMATOLOGICAL TREATMENTS AND SYSTEMS EMPLOYING COOLING, TOPICALS, AND/OR ABRASION”, the entirety of which is hereby incorporated by reference herein.

In some embodiments, cryogen can be sprayed on to skin being treated using systems, devices, and methods as described in U.S. Publication No. 2019-0000524, filed on Jun. 27, 2018, and entitled “Dermatological Cryospray Devices Having Linear Array Of Nozzles And Methods Of Use”, U.S. Publication No. 2020-0214757, filed on Dec. 20, 2019, and entitled “Automated Dermatological Cryospray Treatment Planning System”, U.S. Publication No. 2020-0214757, filed on Dec. 20, 2019, and entitled “Automated Dermatological Cryospray Treatment Planning System”, and U.S. Publication No. 2021-0407201, filed on Jun. 16, 2021, and entitled “Time-Of-Flight (Tof) Camera Systems And Methods For Automated Dermatological Cryospray Treatments”, the entirety of each of which is hereby incorporated by reference herein. In some embodiments, the system can include an automated system that can include robotic control of the spray of cryogen on to the skin being treated.

The cooling treatment system 100 can be used in providing a freeze treatment or a cooling motion treatment. The cooling treatment system 100 can include a body 102, also referred to herein as a housing 102 or as a console 102. The body 102 can house several of the components of the cooling treatment system 100 including, for example, a display 104, a reader 106, a single board computer (“SBC”) 108, and a controller 110. The housing 102 can further connect to a boom 103.

The display 104 can comprise desired display, and can be configured to provide information to the user of the cooling treatment system 100, and in some embodiments, receive inputs from the of the cooling treatment system. In some embodiments, the display can comprise a touch screen.

The reader 106 can comprise a feature configured to read information from an artifact 107. In some embodiments, this artifact 107 can comprise a physical artifact, and in some embodiments, this artifact can comprise a digital token or a non-physical artifact. In some embodiments, the non-physical artifact can, for example, be locally stored or can be remotely stored. In some embodiments, the non-physical artifact can reside on the cloud and/or be accessible via the cloud.

This physical artifact 107 can comprise, for example, any object or token, either physical or digital, comprising computer readable treatment instructions and/or information identifying an associated number of remaining treatment cycles associated with the card. In some embodiments, for example, the number of treatment cycles can track the number of remaining times that the artifact 107 may be used in connection with the cooling treatment system 100 to provide a treatment to a patient. When the number of remaining treatment cycles reaches zero, then the artifact, unless its treatment cycles are reloaded, cannot be used in connection with the cooling treatment system 100 to provide further treatments to patients. Thus, in some embodiments, the artifact comprises a count of available treatments, which count can be decremented when a treatment is provided and which count can be increased if/when the artifact is reloaded. In some embodiments, for example, the artifact can be reloaded by a customer purchase, for example, via the internet. In such an embodiment, subsequent to the successful processing of payment, the value of the count of the artifact can be incremented by the number of purchased treatments.

These instructions can comprise a plurality of step and associated treatments, a plurality of treatment parameter or conditions, or the like. In some embodiments, for example, treatment instructions can be loaded onto an artifact 107, and these instructions can be retrievable from the artifact 107 via the reader 106. In some embodiments, the artifact can comprise, for example, a microprocessor card, also referred to herein as a smart card. The microprocessor card can communicate with the reader 106 via, for example, a contact-based, or contactless communication. In some embodiments, for example, the artifact can include a plurality of contacts, such as found in, for example, a Europay, Mastercard, and Visa chip (EMV chip), which can connect with contacts in the reader 106 to enable reading of the card. In some embodiments, for example, the artifact 107 can contactlessly connect with the reader according to any desired contactless communication standard and/or wireless communication standard. In some embodiments, for example, the artifact 107 can connect to the reader 106 via, for example, NFC, Bluetooth, WiFi, ethernet, or the like.

The SBC 108, which can, for example, comprise a processor, associated memory, drivers, and/or communications buses. The SBC 108 can interface with other components through communications protocols, and specifically can participate in control of some of the components of the cooling treatment system 100, and specifically of the console 102. This can include, for example, the display 104, the reader 106, chiller 211, and/or the antenna 226. In some embodiments, the SBC 108 can support input/output hardware such as, for example, one or several display drivers, interface drivers, USB interface drivers, or the like.

The controller 110, also referred to herein as a processor 110, can comprise, for example, a microprocessor, such as a microprocessor from Intel® or Advanced Micro Devices, Inc.®, or Texas Instrument, or Atmel, or the like. In some embodiments, the controller 110 can comprise a custom electronics board, which can include features for controlling, for example, the chiller 211 and/or cooler 230, and hardware monitoring capabilities.

The controller and/or processor 110 can be communicatively coupled with a memory, which memory can be volatile and/or non-volatile and/or can include volatile and/or non-volatile portions. In some embodiments, the memory can include information and/or instructions, which can be executed by the processor 110. The processor 110 can, according to these instructions, control all or portions of the cooling treatment system 100.

The body 102 can be connected to a handpiece 112 via cord 111, also referred to herein as cable 111. The cord 111 can flexibly couple the handpiece 112 to the body 102. The cord 111 can physically and communicatively connect the handpiece 112 to the body 102 and/or to components contained in the body 102. In some embodiments, for example, the cord 111 can include one or several wires, tubes, optical fiber, or the like. In some embodiments, the controller 110 within the body 102 can control and/or communicate with the handpiece 112 via the cord 111. In some embodiments, coolant from the body 102 can transit to and from the handpiece 112 via the cord. In some embodiments, all or portions of the weight of the cord 111 can be supported by the boom 103.

The handpiece 112 comprises a hand controller 114, a motion detector 115, and a cold plate 116, also referred to herein as a handpiece tip 116, a cold plate 116, a freeze tip 116, and/or a freezing tip 116. The hand controller can comprise, for example, a microprocessor, such as a microprocessor from Intel® or Advanced Micro Devices, Inc.®, or Texas Instrument, or Atmel, or the like.

The hand controller 114 can be communicatively coupled with a memory, which memory can be volatile and/or non-volatile and/or can include volatile and/or non-volatile portions. In some embodiments, the memory can include information and/or instructions, which can be executed by the hand controller 114. The hand controller 114 can, according to these instructions, control all or portions of the handpiece 112, and specifically can control a temperature of the cold plate 116.

The motion detector 115 can be configured to detect motion and/or changes of motion of the handpiece 112. In some embodiments, such as when providing a motion cooling treatment, detection of motion and/or changes of motion of the handpiece 112 can be beneficial in providing a consistent treatment. For example, if the speed with which the handpiece 112 is rubbed across the surface of the skin during a motion cooling treatment changes, then cooling may be unevenly applied to the surface of the skin, which may result in unideal results.

In some embodiments, the motion detector 115 can comprise, for example, an accelerometer which can be configured to detect if/when the speed of the motion of the handpiece 112 changes. In some embodiments, the motion detector 115 can be communicatively coupled to the controller 110 via the cord 111. In some embodiments, and based on information received from the motion detector 115, the controller can provide the user outputs indicative of the speed of the handpiece 112, indicative of changes in speed of the handpiece 112, and/or directing the user accelerate or decelerate the movement of the handpiece 112. In some embodiments, these outputs can be provided to the user via the display 104.

In some embodiments, for example, the controller 110 can receive a signal indicative of a change in motion of the handpiece 112 from the motion detector 115. The controller 110 can determine if this change is sufficiently large as to indicate a meaningful change in the speed of motion of the handpiece 112. In some embodiments, for example, the controller 110 can compare the signal received from the motion detector 115 to one or several thresholds to determine if the change in motion of the handpiece 112 is acceptable or unacceptable. Based on the comparison of the signal to the one or several thresholds, the controller 110 can determine when the motion of the handpiece has unacceptably changed, determine to provide an output, and can provide an output to the user indicating that the motion of the handpiece has unacceptably changed. Based on the comparison of the signal to the one or several thresholds, the controller 110 can determine when the change of motion of the handpiece is acceptable, and based on that, either take no action or provide an output to the user indicating that the motion of the handpiece 112 is acceptable.

The cooling treatment system 100 can further include one or several disposable features which can be contained within a case 118. These features can include one or several tips 119. Specifically, these tips 119 can include a smooth tip 120 and/or a textured tip 122. Each of the smooth tip 120 and the textured tip 122 will be discussed at greater length before. In some embodiments, the case 118 can further include an identifier 124. In some embodiments, the identifier 124 can comprise a SmartChip that can be, for example, located in a lid of the box.

The cooling treatment system 100 can further include one or several topical 132. These one or several topicals 132 can be contained within a case 130. In some embodiments, case 130 can be separate and distinct from case 118, and in some embodiments, case 118 and case 130 can comprise a single case. Case 130 can, in some embodiments, contain the artifact 107 and topicals 130 for providing a treatment. For example, the artifact 107 in the case 130 can include instructions executable by the controller 110 and/or the hand controller 114 to provide the treatment. The case 130 can further include topicals for providing this treatment.

These topicals can include, for example a coupling fluid such as a water-based coupling fluid that can include a water-based gel. The water-based coupling fluid can comprise water, a thickening agent to increase viscosity, and a preservative. The coupling fluid may also include an amount of propylene glycol, alcohol, or similar to depress the freezing point below 0 deg. C. The purpose of the coupling fluid is to control the freezing point of the coupling fluid and therefore influence the freezing point of the skin, improve the heat transfer between the skin and the cold plate and, in the case where the handpiece tip 116 is being swept across the skin, improve the lubricity between the skin and the handpiece tip 116.

In some embodiments, the coupling fluid can include a first coupling fluid configured for use in freeze treatments and a second coupling fluid configured for use in a motion cooling treatment. Each of the first coupling fluid and the second coupling fluid can be configured to lubricate and to improve thermal coupling. The first coupling fluid can be configured to freeze while providing the static freeze treatment, and the second coupling fluid can be configured to remain unfrozen while providing the motion cooling treatment.

In some embodiments, these coupling fluids can include different ingredients and/or have different physical properties. For example, the first coupling fluid configured for use in a freeze treatment can be configured to freeze during the freeze treatment, whereas the second coupling fluid configured for use during the motion cooling treatment can be configured to remain unfrozen during the motion cooling treatment. In some embodiments, and to achieve this effect, the second coupling fluid can include an ingredient configured to suppress the freezing point of the second coupling fluid, whereas the first coupling fluid may not include such an ingredient, or may include less of that ingredient. Thus, in some embodiments, the first coupling fluid can have a freezing point that is higher than the freezing point of the second coupling fluid, in other words, the second coupling fluid can be configured to freeze at lower temperatures than the first coupling fluid.

Further, in some embodiments, the first coupling fluid can have a different viscosity than the second coupling fluid. In some embodiments, for example, the first coupling fluid can have a viscosity of between, for example, approximately 200 centipoise and approximately 400 centipoise, whereas the second coupling fluid can have a viscosity of between, for example, 600 centipoise and approximately 1000 centipoise.

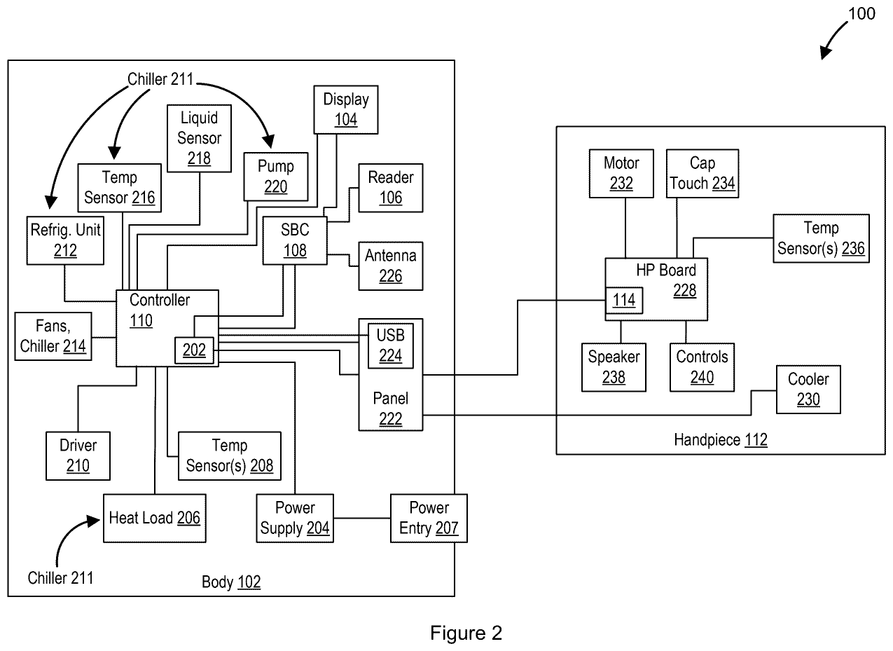

With reference now to FIG. 2, a schematic illustration of electrical components of the cooling treatment system 100 is shown. The body 102 can include controller 110, which can comprise USB chip 202. The USB chip 202 can, convert communications to and from USB signals.

The controller 110 can receive power from power supply 204, which is connected to power entry 207. In some embodiments, power entry 207 can comprise any feature configured to receive power from a source external to the cooling treatment system 100 and deliver that power to the power supply 204. The power entry 207 can comprise, in some embodiments, a power cord or power plug.

The power supply 204 can supply power to the controller 110 and, via the controller 110 to some or all of the powered components of the cooling treatment system 100. The power supply can comprise an AC/DC converter, which can, for example, receive AC power from the power entry 207 and convert that AC power to DC. In some embodiments, the power supply 204 can comprise a 1000 W AC/24V DC converter.

The controller 110 can power and communicate with a heat load 206 and a temperature sensor 208. The heat load 206 can comprise a heater configured to heat a portion of the chiller, and specifically configured to heat coolant traveling through the chiller. The heat load 206 can comprise a resistive heater that can, for example, comprise one or several resistive heating elements. The temperature sensor 208 can be configured to sense a temperature within the body 102, and specifically, a temperature of the heat load 206 and/or of coolant traveling through the heat load 206.

The controller 110 can further power a driver 210, which can drive one or several components of the cooling treatment system 100 including, for example a fan and/or a cooler.

The body 102 can include a chiller 211 which can be configured to chill a coolant to a coolant temperature within a target range. The chiller 211 can include multiple components powered and/or controlled by the controller 110 including, for example, a refrigeration unit 212, a pump 220, and a temperature sensor 216. In some embodiments, the chiller 211 can be cooled by one or several fans 214, which can be powered by the controller 110, and a level of the coolant in the chiller 211 can be detected by liquid sensor 218, which can be a liquid level sensor. In some embodiments, the chiller 211 can comprise a heat exchanger such as for example, a heat exchanger comprising a plurality of fins and a fan configured to blow air across the fins.

The controller 110 can provide regulated DC power to the SBC 108, the display 104, and the panel 222. The SBC 108, can in turn power the reader 106 and the antenna 226, and can communicate with each of the display 104, the reader 106, and the antenna 226. In some embodiments, these communications can comprise, for example, USB signals.

In addition to powering the panel, the controller 110 can maintain a communicating connection with the panel 222 and USB 224. In some embodiments, the panel 222 can comprise an interface through which the cable 111 can connect to the body 102. In some embodiments, for example, the cable 111 can be connected to the panel 222 such that a handpiece board 228, which is a circuit board in the handpiece 112 containing the hand controller 114 is powered and/or communicatively connected to the controller 110 via the cable 111. In some embodiments, via the cable 111, the cooler 230 is powered and/or controlled by the controller 110.

The cooler 230 can be configured to control the cold plate temperature of the cold plate 116. In some embodiments, the cooler 230 can comprise a thermoelectric cooler (TEC), which can transfer heat energy way from the cold plate 116. In some embodiments, the cooler 230 can be configured to transfer this heat energy to a reservoir such as, for example, coolant circulated through the handpiece 112 from the chiller 211.

The handpiece 112 can further include a motor 232, capacitive touch sensor electronics 234, also referred to herein as a cap touch 234, one or several temperature sensors 236, a speaker 238, and one or several input features 240. In some embodiments, each of these components of the handpiece 112 can be powered and/or controlled by the controller 110.

The motor 232 can, in some embodiments be configured to provide haptic feedback and/or tissue vibration. In some embodiments, this vibration can be transferred to the tissue being treated. In some embodiments, these vibrations transferred to the tissue being treated can cause and effect on the tissue being treated. In some embodiments, this can include, for example, causing ice nucleation.

The cap touch 234 can comprise an electrical circuit that is electrically coupled to the cold plate 116. The cap touch 234 can include, for example, a capacitive touch sensor. In some embodiments, the cap touch 234 can be configured, such that the cap touch 234 senses a change in capacitance when the cold plate 116, or one of the tips 119 coupled to the cold plate 116, comes into contact with skin or is removed from contact with the skin. In some embodiments, this can enable an automatic start of treatment by determining when the handpiece 112 contacts the patient's skin or end of treatment as it is removed from the patient's skin. In some embodiments, and as a result to the detection of contact of the handpiece tip 116 with the patient's skin, the countdown time can be triggered.

In some embodiments in which a freeze or a cooling treatment is being provided, the cap touch 234 can detect when the cold plate 116, or one of the tips 119 coupled to the cold plate 116, comes into contact with skin. In such embodiments, the countdown timer can be started for that treatment step when contact is detected. In some embodiments in which dwell time is being provided, the cap touch 234 can detect when the cold plate 116, or one of the tips 119 coupled to the cold plate 116, is removed from contact with skin. In such an embodiment, the countdown timer can be started for the dwell time when the removal from contact is detected.

The temperature sensor(s) 236 can be configured to sense one or several temperatures in the handpiece 112. This can include, for example, sensing one or several temperatures of the cold plate 116 and/or one or several temperatures of the handpiece 112 such as, for example, a temperature of the cooler 230. In some embodiments, based on the sensed temperatures, the cooler 230 can increase or decrease its cooling of the cold plate 116.

The handpiece 112 further includes the speaker 238 and controls 240. The speaker 238 can comprise an output device configured to provide information to the user of the cooling treatment system 100. The controls 240 can comprise one or several input features, and specifically one or several buttons. In some embodiments, the user can manipulate the controls 240 to provide one or several inputs to the cooling treatment system 100, and the cooling treatment system 100 can provide information to the user via the speaker 238.

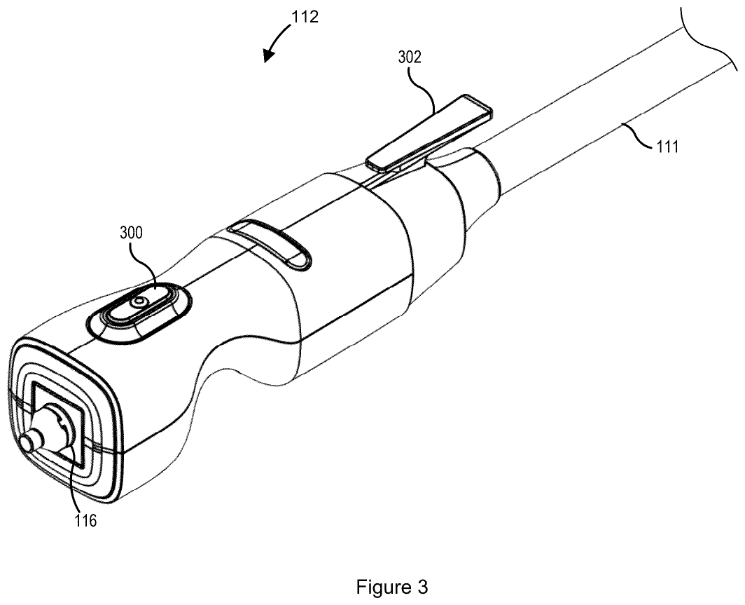

With reference now to FIG. 3, a perspective view of one embodiment of the handpiece 112 is shown. As seen in FIG. 3, the handpiece 112 connects to the cable 111. The handpiece 112 include the cold plate 116. The cold plate 116 can be controllably cooled to enable the delivery of a cooling and/or freeze treatment.

The handpiece 112 further includes an input feature 300. In some embodiments, and as depicted in FIG. 3, this input feature can comprise a button. The handpiece 112 can further include a retention connector 302.

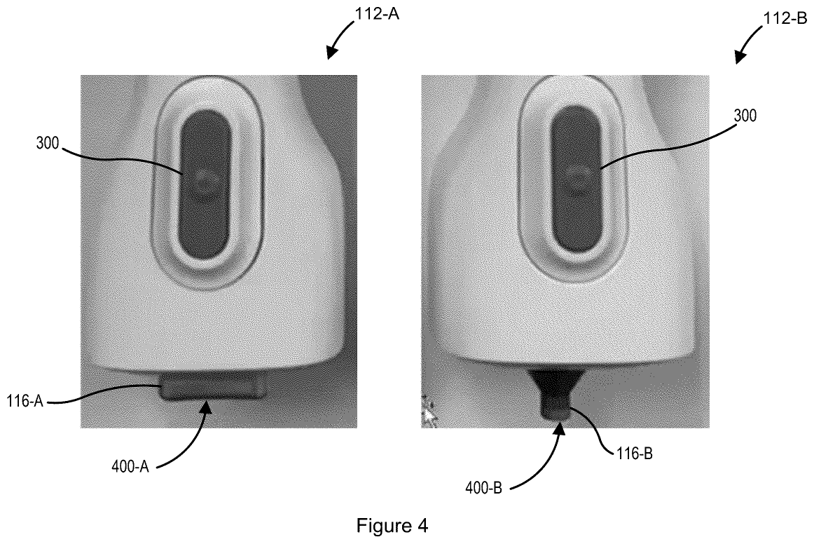

With reference now to FIG. 4, front views of portions of two handpieces 112 are shown. These handpieces 112 include a first handpiece 112-A and a second handpiece 112-B. As depicted, each of these handpieces 112-A, 112-B includes a cold plate 116 and an input feature 300. The cold plate 116-A of the first handpiece 112-A, and specifically, a distal tip 400-A of the cold plate 116-A comprises a square. The square cold plate 116-A of the first handpiece 112-A can have a length and wide of, for example, between approximately 5 mm and approximately 50 mm, between approximately 10 mm and approximately 40 mm, between approximately 20 mm and 30 mm, of approximately 7 mm, of approximately 25 mm, or any other or intermediate dimension.

The cold plate 116-B of the second handpiece 112-B, and specifically, a distal tip 400-B of the cold plate 116-B comprises a circle. The circular cold plate 116-B of the second handpiece 112-B can have a diameter of, for example, between approximately 5 mm and approximately 50 mm, between approximately 10 mm and approximately 40 mm, between approximately 20 mm and 30 mm, of approximately 7 mm, of approximately 25 mm, or any other or intermediate diameter.

In some embodiments, the cold plate 116, and specifically the distal end 400 of the cold plate 116 can be directly applied to a patient's skin to provide a cooling and/or freeze treatments. In some embodiments, one or several treatment tips 119, also referred to herein as treatment caps 119 can be coupled to the cold plate 116 and directly applied to the patient's skin to provide a treatment that can include, for example, a dermabrasion treatment, a cooling treatment, and/or freeze treatment.

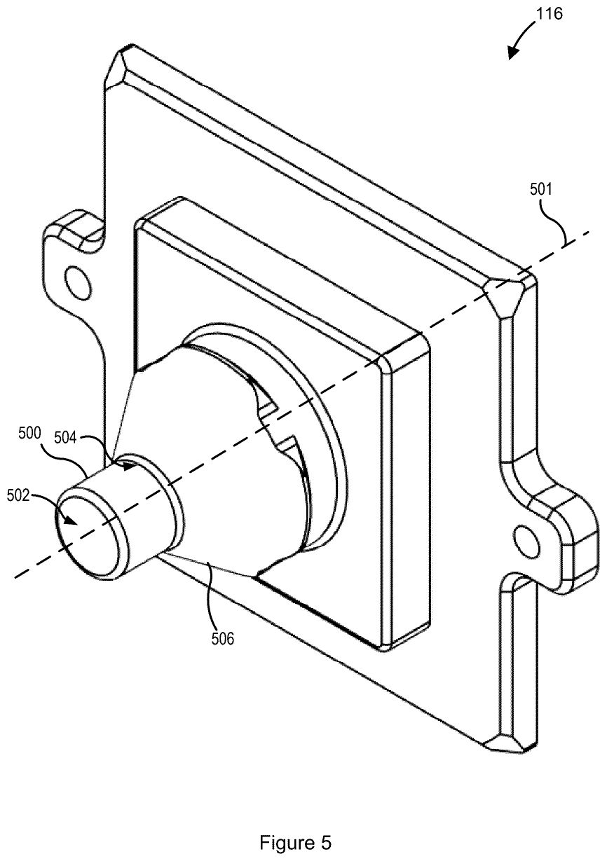

With reference now to FIG. 5, a perspective view of the cold plate 116 is shown. The cold plate 116 includes a distal protrusion 500. The distal protrusion can comprise a cylindrical member extending along axis 501, and radially around axis 501. The distal protrusion 500 can comprise a distal end 502 and a proximal end 504. The distal end 502 can comprise a planar member that can, in some embodiments, extend perpendicular to the axis 501.

The proximal end 504 of the distal protrusion 500 can be coupled to a camming surface 506. The camming surface 506 can extend proximally and radially away from the proximal end 504 of the distal protrusion 500. The camming surface 506 can define a truncated cone, specifically, the camming surface comprising the frustum of the truncated cone. Thus, in some embodiments, the camming surface 506 is at least partially conical.

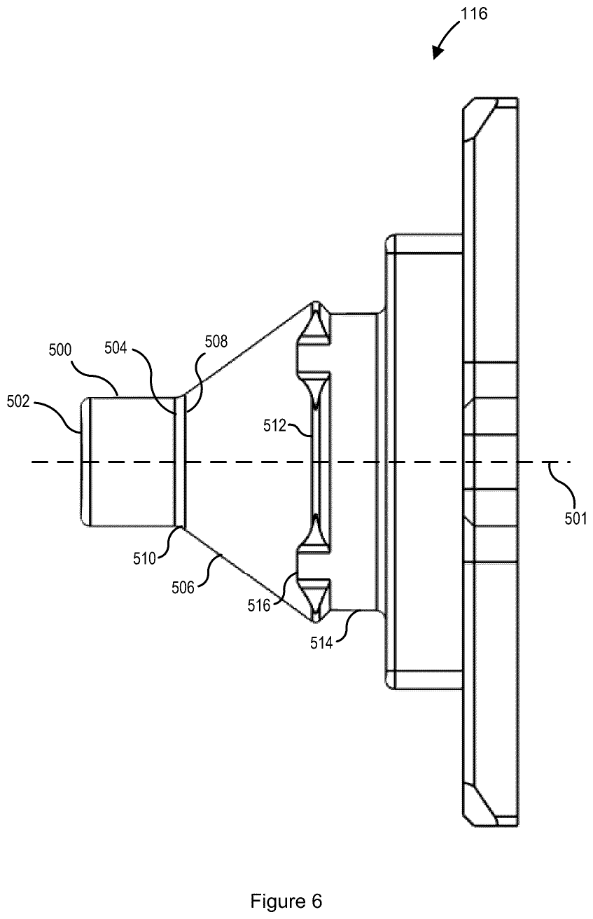

As seen in FIG. 6, a side view of the cold plate 116, the camming surface 506 can have a distal end 508 which can connect directly or indirectly to the proximal end 504 of the distal protrusion 500. In the embodiment of FIG. 6, the distal end 508 of the camming surface 506 connects to the proximal end 504 of the distal protrusion via smoothing feature 510, which can comprise, for example, a blend or round. The camming surface 506 can further comprise a proximal end 512.

The cold plate 116 can further comprise a retention depression 514 extending proximally and radially inward from the proximal end 512 of the camming surface 506. Thus, a diameter of at least a portion of the retention depression 514 measured from the axis 501 can be less than a diameter of the proximal end 512 of the camming surface 506. In other words, the retention depression 514 can have a depth below the proximal end 512 of the camming surface 506.

In some embodiments, the cold plate 116 can further comprise a plurality of axial grooves 516 spread around the circumference of the proximal end 512 of the camming surface. In some embodiments, each of these axial grooves 516 can be located at a junction of the proximal end 512 of the camming surface 506 and the retention depression 514. In some embodiments, each of the axial grooves 516 can extend into the proximal end 512 of the camming surface 506 to a depth equal to a depth of the retention depression 514.

In some embodiments, the cold plate 116, and specifically the distal end 502 of the distal protrusion 500 can be directly applied to a patient's skin to provide a cooling and/or freeze treatments. In some embodiments, one or several treatment tips 119, also referred to herein as treatment caps 119 can be coupled to the cold plate 116 and directly applied to the patient's skin to provide a treatment that can include, for example, a dermabrasion treatment, a cooling treatment, and/or freeze treatment.

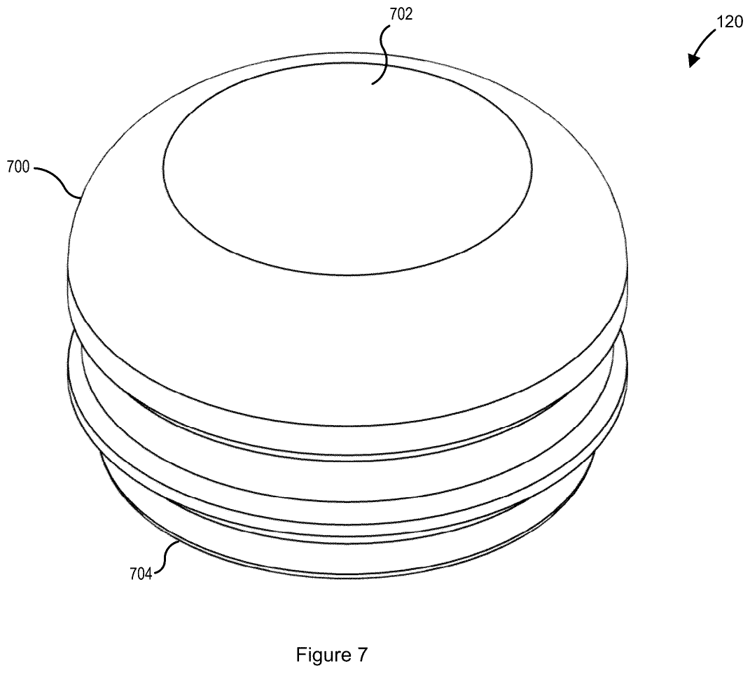

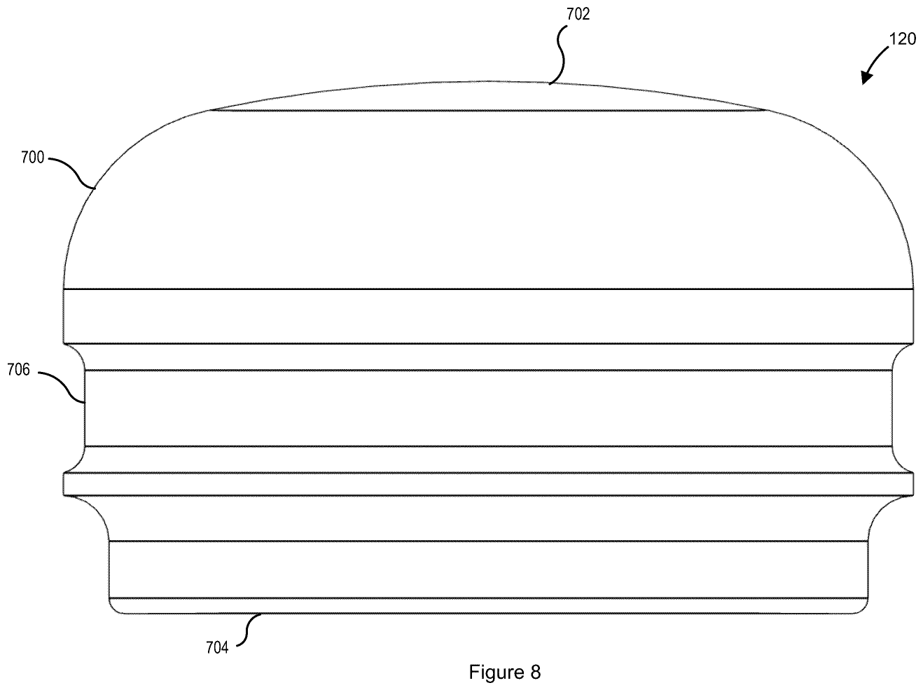

With reference now to FIG. 7, a perspective view of one embodiment of a smooth tip 120 is shown. The smooth tip 120 can include an exterior housing 700, a contact surface 702, and a base 704. The contact surface 702 can comprise a variety of shapes and sizes. In some embodiments, the contact surface 702 can comprise a circular surface that can comprise a diameter of, for example, between 5 mm and 40 mm, between 10 mm and 30 mm, or any other or intermediate diameter. In some embodiments, the contact surface 702 can comprise a square or rectangular surface that can comprise a length and/or width between 5 mm and 40 mm, between 10 mm and 30 mm, or any other or intermediate sizes. In some embodiments, the contact surface 702 of the smooth tip 120 can be smooth. In some embodiments, the smooth tip 120 can have a roughness Rz of between 5 and 50 microns, between 10 and 40 microns, between 20 and 30 microns, approximately 25 microns +/−10 microns, approximately 25 microns +/−5 microns, or any other or intermediate value. As seen in FIG. 8, a side view of the smooth tip 120, the contact surface 702 can, in some embodiments, be curved. The exterior housing 700 of the smooth tip 120 can define a recess 706 extending around housing 700.

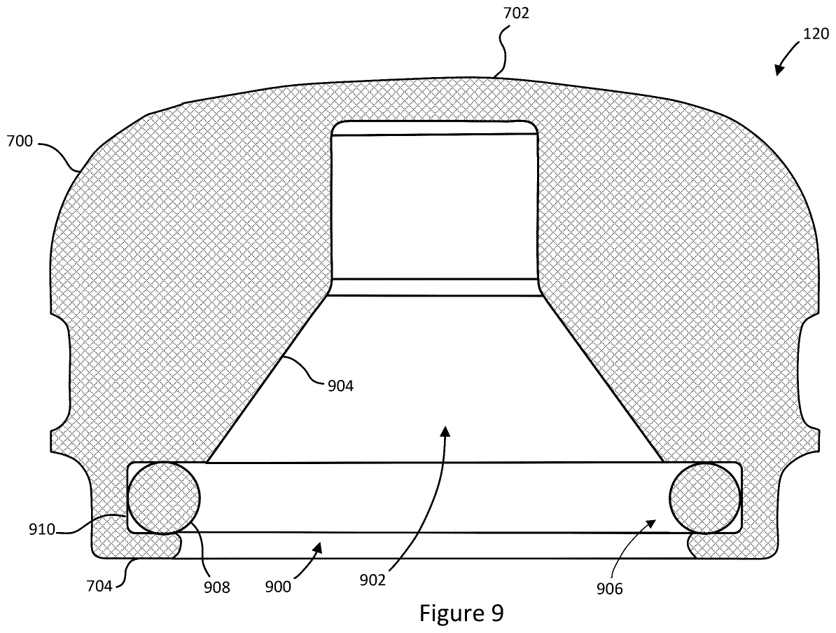

With reference now to FIG. 9, a section view of the smooth tip 120 is shown. As seen in FIG. 9, the housing 700 can define an opening 900 in the base 704 of the housing 700, which opening can extend into a receptacle 902 defined by the housing 700. The receptacle 902 can be configured to receive the cold plate 116. The receptacle 902 can be defined by mating surfaces 904 having wholly or partially the same shapes as portions of the cold plate 116, and specifically of the distal protrusion 500 and the camming surface 506. These mating surfaces 904 can be configured to mate with and/or directly or indirectly contact with the cold plate 116, and specifically with the distal protrusion 500 and/or the camming surface 506 to facilitate heat transfer from the smooth tip 120 to the cold plate 116. In some embodiments, the mating surfaces 904 can perfectly mate with the cold plate 116 and specifically with the distal protrusion 500 and/or the camming surface 506 such that the mating surfaces 904 physically contact the distal protrusion 500 and/or the camming surface 506. However, such perfect mating may not be achieved, and a small gap may exist between all or portions of the mating surfaces 904 and the corresponding portions of the cold plate 116 and specifically of the distal protrusion 500 and/or the camming surface 506. In some embodiments, the smooth tip 120 can be thermally coupled to the cold plate 116 via these mating surfaces 904, and in some embodiments via a thermally conductive coupling fluid applied to the mating surfaces 904 and/or to the cold plate 116. The thermally conductive fluid can, in some embodiments, fill some or all of the gaps between the mating surfaces 904 and the cold plate 116 and specifically the distal protrusion 500 and/or the camming surface 506. In some embodiments, this thermally conductive fluid can improve thermal coupling between the smooth tip 120 and the cold plate 116. This improved thermal coupling can in some embodiments, improve and/or enhance the control of the temperature of the smooth tip 120.

The thermally conductive fluid can be a coupling fluid as disclosed herein. The coupling fluid can be, for example, a water-based coupling fluid such as a water-based gel. The water-based coupling fluid can, for example, comprise water and a thickening agent. In some embodiments, the coupling fluid can comprise between approximately 90% and approximately 99% water by weight, and/or can comprise approximately 98% water by weight. In some embodiments, the coupling fluid can comprise between approximately 10% and approximately 1% thickening agent by weight, and/or can comprise approximately 2% thickening agent by weight.

The coupling fluid may also include an amount of propylene glycol, alcohol, or similar to depress the freezing point below 0 deg. C. In some embodiments, for example, the coupling fluid can better thermally couple the cold plate 116 and one of the tips 119 such as the smooth tip 120 when in liquid form. Thus, in some embodiments, the addition of a freeze-point depressing substance to the coupling fluid can improve the performance of the coupling fluid.

In some embodiments, the thermally conductive fluid can be configured for easy cleanup after use. Specifically, for example, the thermally conductive fluid can be water-based as opposed to oil-based, and/or the thermally conductive fluid can be free of thermally conductive particles.

Surprisingly, it was determined that a water-based coupling fluid provides advantages as compared to other thermal coupling substances, such as thermal pastes. Specifically, a water-based coupling fluid provides good thermal coupling while being significantly easier to clean off of a cold plate 116 and/or a tip 119. Specifically, a water-based coupling fluid that is water-soluble can be washed off of the cold plate 116 and/or tip 119, thereby cleaning the cold plate 116 and/or tip 119.

The smooth tip 120, and specifically, the housing 700 can comprise a retention feature 906, which can, in some embodiments, comprise a compliant member coupled to the housing 700. As specifically depicted in FIG. 9, the retention feature 906 comprises a compliant member in the form of an O-ring 908, such as a rubber O-ring, that is retained in a recess 910 defined by the housing 700. In some embodiments, this compliant member, and specifically O-ring 908, can be configured to be received and retained within the retention depression 514 of the cold plate 116 when the smooth tip 120 is coupled to the cold plate 116. As will be discussed below, all of tips 119 can include similar retention features to allow those tips 119 to be coupled to the cold plate 116.

In some embodiments, for example, when the smooth tip 120 is coupled to the cold plate 116, the compliant member, and specifically the O-ring 908, can engage with the camming surface 506, which can deform, and specifically can expand the diameter of the compliant member, and specifically the O-ring 908. This deformation of the compliant member can occur until the cold plate 116 has been sufficiently inserted into the receptacle 902 of the housing 700 of the smooth tip 120 such that the compliant member, more specifically the O-ring 908, is received within the retention depression 514. Due to the smaller diameter of the retention depression 514, the compliant member can return to its undeformed shape or to a less deformed shape. This can include the decreasing of the diameter of the O-ring. This return to an undeformed or to a less deformed shape retains the smooth tip 120 on the cold plate 116. The smooth tip 120 can be decoupled from the cold plate 116 by applying sufficient force to extract the compliant member from the retention depression 514.

In some embodiments, axial grooves 516 can facilitate the coupling and decoupling of the smooth tip from the cold plate. Specifically, these axial grooves 516 prevent the sealing of the compliant member, and specifically the O-ring 908 around the cold plate 116. Preventing this sealing likewise prevents the creation of a vacuum when the smooth tip 120 is removed from the cold plate 116 or the creation of pressurized gas or fluid when the smooth tip 120 is coupled to the cold plate 116.

In some embodiments, the coupling fluid can be applied to all or portions of the cold plate 116 and/or into the receptacle 902 to all or portions of the mating surfaces 904. When the tip 110 is applied to the cold plate, the O-ring 908 can seal around the cold plate 116. However, due to the axial grooves 516, air and/or excess coupling fluid can be pushed out from between the cold plate 116 and the mating surfaces 904 when the tip 119 is attached to the cold plate 116. Alternatively, when the tip 119 is removed from the cold plate 116, the axial grooves can allow air and/or coupling fluid to enter between the cold plate 116 and the tip 119. This movement of fluid and/or air into and/or out of the space between the cold plate 116 and the tip 119 can facilitate the coupling of the tip 119 to the cold plate 116 and/or the removal of the tip 119 from the cold plate 116 via prevention of the buildup of hydraulic pressure preventing the securing of the tip 119 to the cold plate 116 when the tip 119 is coupled to the cold plate 116 or the creation of a vacuum preventing the separation of the tip 119 from the cold plate 116.

In some embodiments, for example, absence of the axial grooves 516 can result in the entrapment of air and/or fluid between the cold plate and a treatment tip 119. The entrapped air and/or fluid can provide several disadvantages. Specifically, in embodiments in which the treatment tip 119 is thermally connected to the cold plate to enable the providing of cooling and/or freeze treatment, this entrapped air can decrease the thermal coupling between the cold plate and the treatment tip. Further, such entrapped air and/or fluid can decrease the stability of the connection of the treatment tip 119 to the cold plate.

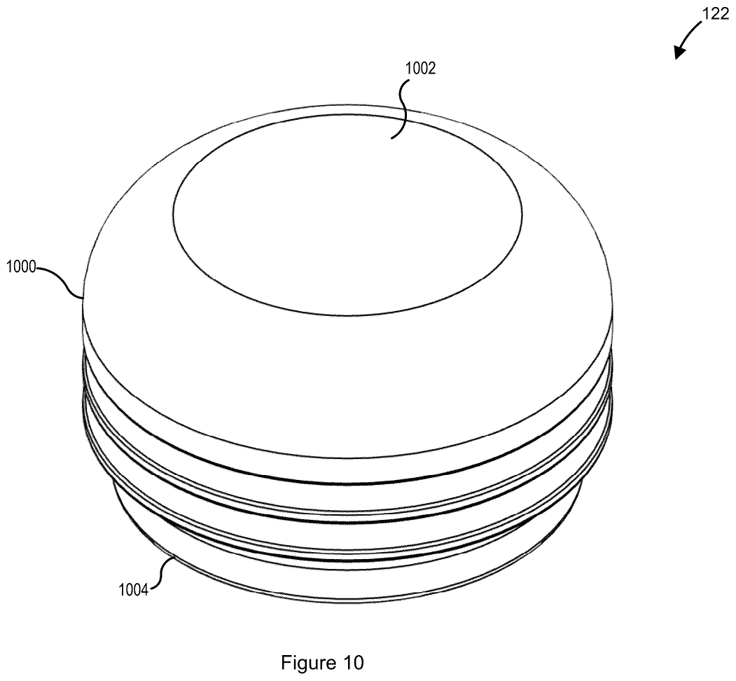

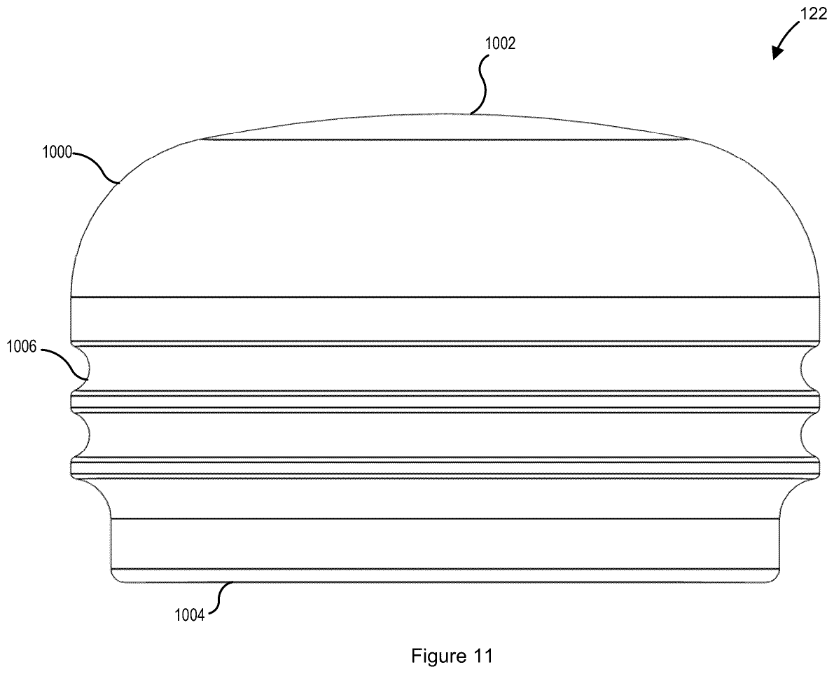

FIGS. 10 and 11 shows views of an embodiment of a tip 119, and specifically of a textured tip 122. In some embodiments, the textured tip 122 can be sufficiently textured so as to abrade skin when the textured tip 122 is rubbed on the skin.

As seen in FIG. 10, a perspective view of the textured tip 122 can include an exterior housing 1000, a contact surface 1002, and a base 1004. The contact surface 1002 can be flat, planar, non-planar, dimpled, concave, or convex. The contact surface 1002 can comprise a variety of shapes and sizes. In some embodiments, the contact surface 1002 can comprise a circular surface that can comprise a diameter of, for example, between 5 mm and 40 mm, between 10 mm and 30 mm, or any other or intermediate diameter. In some embodiments, the contact surface 1002 can comprise a square or rectangular surface that can comprise a length and/or width between 5 mm and 40 mm, between 10 mm and 30 mm, or any other or intermediate sizes. In some embodiments, the contact surface 1002 of the textured tip 122 can be textured. In some embodiments, the textured tip 122 can have a roughness Rz of between 50 and 270 microns, between 60 and 200 microns, between 70 and 150 microns, approximately 100 microns +/−20 microns, approximately 100 microns +/−10 microns approximately 100 microns +/−5 microns, approximately 80 microns +/−10 microns, approximately 80 microns +/−5 microns, approximately 75 microns +/−10 microns, approximately 75 microns +/−5 microns, or any other or intermediate value. As seen in FIG. 8, a side view of the textured tip 122, the contact surface 1002 can, in some embodiments, be curved. The exterior housing 1000 of the textured tip 122 can define a plurality of recesses 1006 extending around housing 1000. In some embodiments, the smooth tip 120 and the textured tip 122 can have a different number of recesses 706, 1006 to enable easy visual or tactile differentiation between the smooth tip 120 and the textured tip 122.

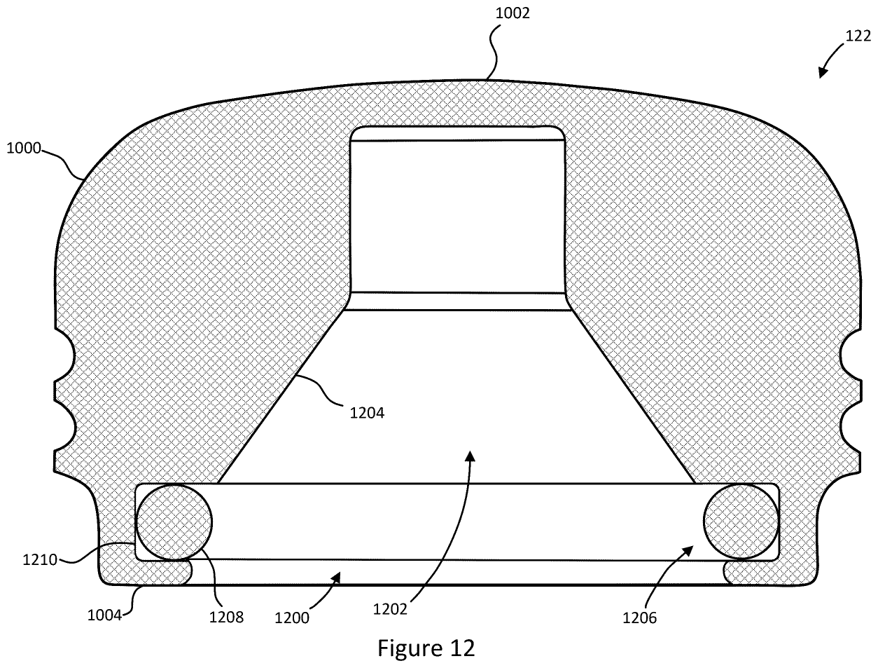

With reference now to FIG. 12, a section view of the textured tip 122 is shown. As seen in textured tip 122, the housing 1000 can define an opening 1200 in the base 1004 of the housing 1000, which opening can extend into a receptacle 1202 defined by the housing 1000. The receptacle 1202 can be configured to receive the cold plate 116. The receptacle 1202 can be defined by mating surfaces 1204 having wholly or partially the same shapes as portions of the cold plate 116, and specifically of the distal protrusion 500 and the camming surface 506. These mating surfaces 1204 can be configured to mate with and/or directly or indirectly contact with the cold plate 116, and specifically with the distal protrusion 500 and/or the camming surface 506 to facilitate heat transfer from the textured tip 122 to the cold plate 116. In some embodiments, the textured tip 122 can be thermally coupled to the cold plate 116 via these mating surfaces 1204, and in some embodiments via a thermally conductive coupling fluid applied to the mating surfaces 1204 and/or to the cold plate 116.

In some embodiments, the mating surfaces 1204 can perfectly mate with the cold plate 116 and specifically with the distal protrusion 500 and/or the camming surface 506 such that the mating surfaces 1204 physically contact the distal protrusion 500 and/or the camming surface 506. However, such perfect mating may not be achieved, and a small gap may exist between all or portions of the mating surfaces 1204 and the corresponding portions of the cold plate 116 and specifically of the distal protrusion 500 and/or the camming surface 506. In some embodiments, the textured tip 122 can be thermally coupled to the cold plate 116 via these mating surfaces 1204, and in some embodiments via a thermally conductive coupling fluid applied to the mating surfaces 1204 and/or to the cold plate 116. The thermally conductive fluid can, in some embodiments, fill some or all of the gaps between the mating surfaces 1204 and the cold plate 116 and specifically the distal protrusion 500 and/or the camming surface 506. In some embodiments, this thermally conductive fluid can improve thermal coupling between the textured tip 122 and the cold plate 116. This improved thermal coupling can in some embodiments, improve and/or enhance the control of the temperature of the textured tip 122.

The thermally conductive fluid can be a coupling fluid as disclosed herein. The coupling fluid can be, for example, a water-based coupling fluid such as a water-based gel. The water-based coupling fluid can, for example, comprise water and a thickening agent. In some embodiments, the coupling fluid can comprise between approximately 90% and approximately 99% water by weight, and/or can comprise approximately 98% water by weight. In some embodiments, the coupling fluid can comprise between approximately 10% and approximately 1% thickening agent by weight, and/or can comprise approximately 2% thickening agent by weight.

The coupling fluid can also include an amount of propylene glycol, alcohol, or similar to depress the freezing point below 0 deg. C. In some embodiments, for example, the coupling fluid can better thermally couple the cold plate 116 and one of the tips 119 such as the smooth tip 120 when in liquid form. Thus, in some embodiments, the addition of a freeze-point depressing substance to the coupling fluid can improve the performance of the coupling fluid.

Additionally, in some embodiments, the coupling fluid can include at least one antimicrobial substance.

In some embodiments, the thermally conductive fluid can be configured for easy cleanup after use. Specifically, for example, the thermally conductive fluid can be water-based as opposed to oil-based, and/or the thermally conductive fluid can be free of thermally conductive particles.

Surprisingly, it was determined that a water-based coupling fluid provides advantages as compared to other thermal coupling substances, such as thermal pastes. Specifically, a water-based coupling fluid provides good thermal coupling while being significantly easier to clean off of a cold plate 116 and/or a tip 119. Specifically, a water-based coupling fluid that is water-soluble can be washed off of the cold plate 116 and/or tip 119, thereby cleaning the cold plate 116 and/or tip 119.

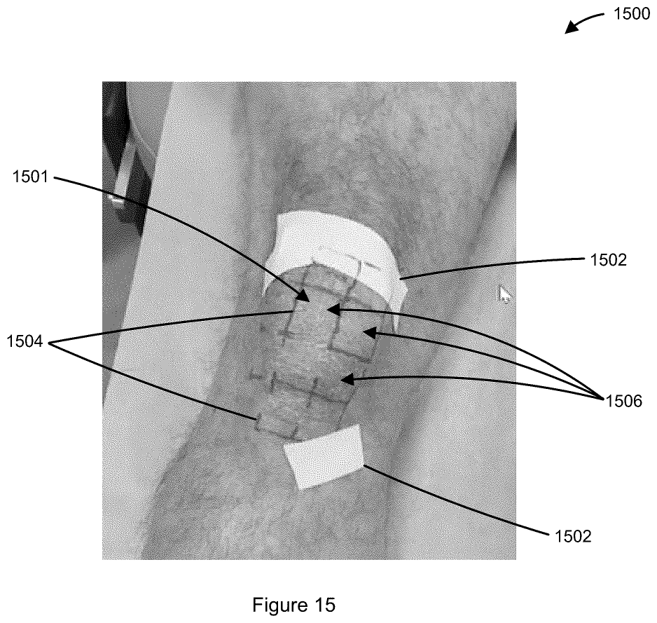

The textured tip 122, and specifically, the housing 1000 can comprise a retention feature 1206, which can, in some embodiments, comprise a compliant member coupled to the housing 1000. As specifically depicted in FIG. 15, the retention feature 1206 comprises a compliant member in the form of an O-ring 1208, such as a rubber O-ring, that is retained in a recess 1210 defined by the housing 1000. In some embodiments, this compliant member, and specifically O-ring 1208, can be configured to be received and retained within the retention depression 514 of the cold plate 116 when the textured tip 122 is coupled to the cold plate 116.

In some embodiments, for example, when the textured tip 122 is coupled to the cold plate 116, the compliant member, and specifically the O-ring 1208, can engage with the camming surface 506, which can deform, and specifically can expand the diameter of the compliant member, and specifically the O-ring 1208. This deformation of the compliant member can occur until the cold plate 116 has been sufficiently inserted into the receptacle 1202 of the housing 1000 of the smooth tip 120 such that the compliant member, more specifically the O-ring 1208, is received within the retention depression 514. Due to the smaller diameter of the retention depression 514, the compliant member can return to its undeformed shape or to a less deformed shape. This can include the decreasing of the diameter of the O-ring. This return to an undeformed or to a less deformed shape retains the textured tip 122 on the cold plate 116. The textured tip 122 can be decoupled from the cold plate 116 by applying sufficient force to extract the compliant member from the retention depression 514.

In some embodiments, axial grooves 516 can facilitate the coupling and decoupling of the smooth tip from the cold plate. Specifically, these axial grooves 516 prevent the sealing of the compliant member, and specifically the O-ring 1208 around the cold plate 116. Preventing this sealing likewise prevents the creation of a vacuum when the textured tip 122 is removed from the cold plate 116 or the creation of pressurized gas or fluid when the textured tip 122 is coupled to the cold plate 116.

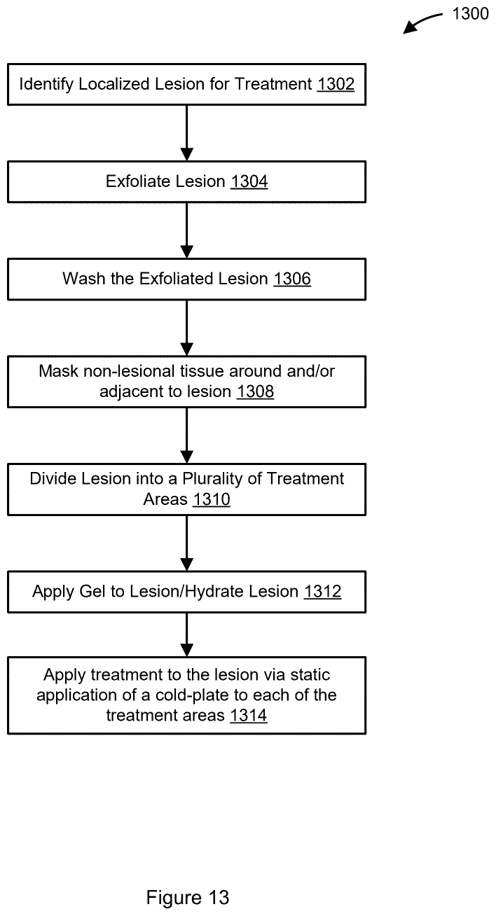

With reference now to FIG. 13, a flowchart illustrating one embodiment of process 1300 for performing a static freeze treatment is shown. The process 1300 can be performed utilizing a system such as a cooling treatment system 100. In some embodiment, the process 1300 can be performed to treat localized lesions, and specifically to treat localized psoriatic lesions. In some embodiments, the process 1300 can be repeated multiple times in treating a lesion. In some embodiments, for example, the process 1300 can be performed once, twice, three times, four times, five times, six times, seven times, eight times, nine times, ten times, fifteen times, twenty times, or any other or intermediate number of times to treat the lesion.

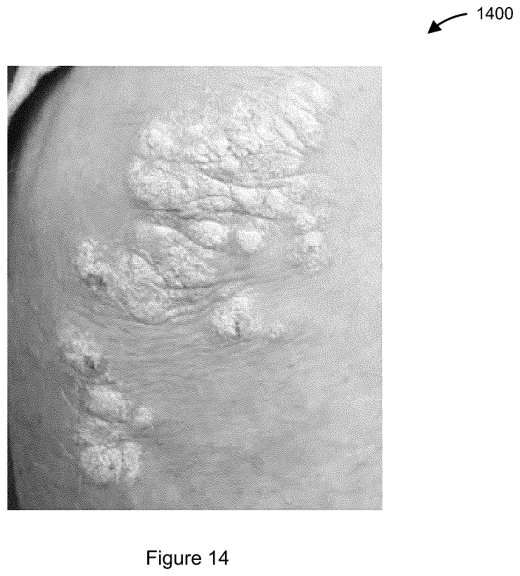

The process 1300 begins at block 1302, wherein a localized lesion is identified for treatment. In some embodiments, the localized lesion can comprise a psoriatic lesion, such as, for example, a psoriatic plaque. In some embodiments, a localized lesion can be a lesion that has an area less than approximately the size of a hand including the palm and the fingers, and specifically has an area of less than approximately 400 cm² to approximately 450 cm². One embodiment of a localized lesion 1400 is shown in FIG. 14, which localized lesion comprises a psoriatic plaque on the knee of a patient.

If a lesion is identified as a localized lesion, then the process 1300 proceeds to block 1304, wherein the lesion is exfoliated. In some embodiments, the lesion can be exfoliated to remove any dead and/or loose tissue from the lesion to thereby facilitate thermal conductivity through the lesion and/or facilitate the propagation of ice through the lesion. In some embodiments, the lesion can be mechanically exfoliated or chemically exfoliated. In some embodiments, the exfoliation can be performed via an abrading device such as the textured tip 122, or via applying and removing an adhesive material to the plaque. For example, tape, such as waterproof tape can be applied to and removed from the plaque, thereby removing loose material from the plaque which loose material adheres to the tape.

As indicated in block 1306, upon completion of the exfoliation, the area to be treated, and specifically, the exfoliated area can be washed and/or cleansed to remove any debris left from the exfoliation. In some embodiments, the removal of the debris left from the exfoliation can improve the thermal conductivity and/or ice propagation through the lesion. This washing and/or cleansing can be performed with water, alcohol, a cleanser, or the like. In some embodiments, water or alcohol can be used for the washing as neither leaves a residue upon drying and/or evaporating. In embodiments in which a cleanser is used, the cleanser can be selected to minimize any residue left subsequent to drying and/or evaporating.

At block 1308, non-lesional tissue around and/or adjacent to the lesion can be masked, or in other words, can be covered to prevent freezing of that tissue. In some embodiments, the masking can include applying a waterproof material to the non-lesional tissue around and/or adjacent to the lesion. This waterproof material can block the propagation of ice through the lesion and into the underlying skin. In some embodiments, this waterproof material can comprise, for example, a waterproof tape and/or an elastomer. In some embodiments, the waterproof tapes, such as, for example, a medical and/or athletic tape, can be adhered to the healthy and/or non-lesional tissue around and/or adjacent to the lesion. In some embodiments, an elastomer can be applied, via for example, brushing, smearing, pouring, and/or spraying the elastomer on to the healthy and/or non-lesional tissue around and/or adjacent to the lesion.

At block 1310, the lesion can be divided into a plurality of treatment areas. In some embodiments, each of these treatment areas can have the same size and/or the same dimensions. In some embodiments, each of these treatment areas can be individually treated, and treating each of the individual treatment areas leads to treating the entirety of the lesion.

With reference now to FIG. 15, a patient's knee 1500 is shown. The knee 1500 includes a lesion 1501, which comprises a psoriatic plaque. The lesion 1501 has been exfoliated and washed subsequent to the exfoliation. Additionally, non-lesional tissue surrounding the lesion has been masked by masking 1502, which masking 1502 comprises waterproof tape adhered to the non-lesional tissue. Further, grid 1504 has been applied to the patient's skin, the grid 1504 defining a plurality of approximately equally sized treatment areas 1506, And specifically defines 12 approximately 12 equally sized treatment areas 1506.

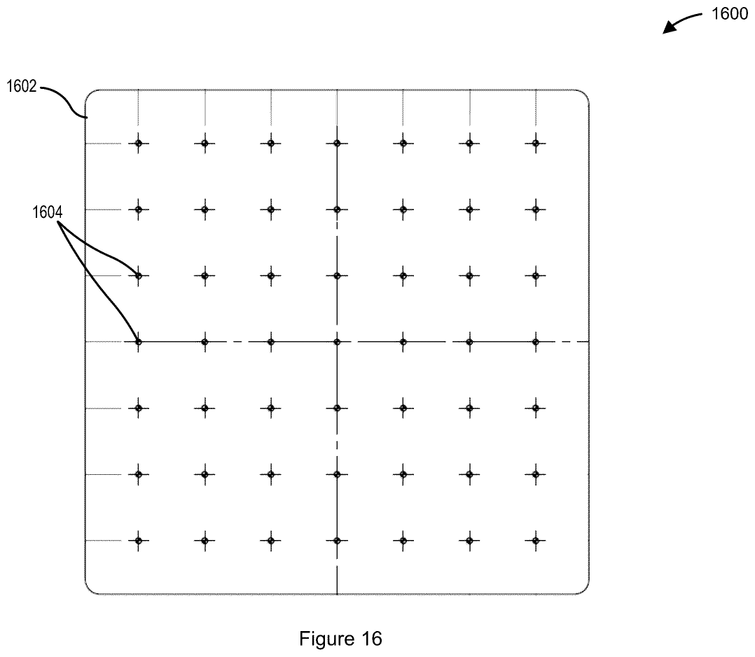

With reference to FIG. 16, in some embodiments, the lesion can be divided into a plurality of treatment areas by grid 1504 applied via a template 1600. This template 1600 can comprise, for example, a flexible, planar member 1602 that can define a plurality of holes 1604. In some embodiments, the skin of and/or around a lesion can be marked through some or all of the plurality of holes 1604 to apply the grid to the lesion. In some embodiments, the template 1600 can be sufficiently flexible as to bend around contours of the patient's anatomy such that the grid can be applied to a non-planar surface on the patient's body. In some embodiments, the template 1600 can be sufficiently flexible to conform to skin surfaces, and specifically to conform to three-dimensional convex skin surfaces such as found, for example, on joints like at the elbow.

In some embodiments, the template 1600 can include any desired number of holes 1604. These holes 1604, can in some embodiments, comprise equally spaced and/or non-equally spaced holes 1604. In some embodiments, the template 1600 can define holes 1604 having a spacing such that the grid generated via use of the template 1600 defines treatment areas that, in some embodiments, are smaller than the cold plate 116, or in other words, the treatment areas have one or several dimensions that a smaller than one or several corresponding dimensions of the cold plate 116. Thus, in some embodiments, the holes 1604 of the template 1600 have an equal spacing that is less than a dimension of the cold plate 116.

In some embodiment, a template 1600 can include between 10 and 100 equally spaced holes 1604, between 25 and 75 equally spaced holes 1604, approximately 49 equally spaced holes 1604, or any other or intermediate number of equally spaced holes 1604. In some embodiments, applying the grid to the area to be treated can include positioning the template 1600 on the plaque, deforming the flexible, planar member 1602 such that the shape of the planar member 1602 corresponds to the shape of the area to be treated, and marking the patient's skin through the holes 1604 in the template 1600.

At block 1312, the lesion is hydrated and/or coupling fluid is applied to the lesion. In some embodiments hydrating the lesion can include applying water to the plaque for a period of time. In some embodiments, this water can be applied directly to the lesion, and in some embodiments, a carrier containing this water can be applied to the lesion. In some embodiments, for example, a wet cloth or gauze can be applied to the lesion to thereby hydrate the lesion. In some embodiments, this wet cloth or gauze can be applied to the lesion for a desired period of time, which desired period of time can be, for example, between approximately 1 minute an approximately 5 minutes, between approximately 2 minutes and approximately 3 minutes, or any other or intermediate period of time. In some embodiments, the wet cloth or gauze can be applied to the lesion for a relatively longer time as a thickness of the lesion increases.

In some embodiments, the coupling fluid can hydrate the tissue of the area to be treated and facilitate the propagation of ice crystals through the area to be treated and into the underlying tissue. In some embodiments in which the area to be treated comprises a localized plaque, and specifically a localized psoriatic plaque, the coupling fluid can be applied to the localized plaque and can hydrate the localized plaque and facilitate in the propagation of ice crystals into and through the plaque and into the skin underneath the plaque.

In some embodiments, the coupling fluid or water can fill in air pockets and gaps in the area to be treated. Specifically, in some embodiments, the coupling fluid can fill air pockets and/or gaps in the lesion skin (e.g., resulting air pockets after the removal of debris and dead cells during the exfoliation step) and can help promote consistent and effective freeze of the plaque.

In some embodiments, the coupling fluid can be a water-based coupling fluid that can, for example, comprise water and a thickening agent. In some embodiments, the coupling fluid can comprise between approximately 90% and approximately 99% water, and/or can comprise approximately 98% water. In some embodiments, the coupling fluid can comprise between approximately 10% and approximately 1% thickening agent, and/or can comprise approximately 2% thickening agent.

In some embodiments, the coupling fluid can comprise water, a substance to either increase or decrease the freezing point of the coupling fluid such as, for example, propylene glycol, a thickening agent such as, for example, sodium carboxymethylcellulose, and/or a preservative. In some embodiments, for example, the coupling fluid can comprise between approximately 30% and approximately 90% water, approximately 60% water, or any other or intermediate percent. In some embodiments, the coupling fluid can comprise between approximately 20% and approximately 60% substance to increase or decrease the freezing point of the coupling fluid, approximately 40% substance to increase or decrease the freezing point of the coupling fluid, or any other or intermediate percent of substance to increase or decrease the freezing point of the coupling fluid. In some embodiments, the coupling fluid can comprise between approximately 1% and approximately 3% thickening agent, approximately 2% thickening agent, or any other or intermediate percent of thickening agent.

The coupling fluid can be configured to have a viscosity sufficient to allow the coupling fluid to penetrate into the plaque without running off of the plaque. In some embodiments, this can be a viscosity of between, for example, approximately 200 centipoise and approximately 400 centipoise. In some embodiments, the coupling fluid can have a pH or between approximately 5.0 and approximately 7.5.

In some embodiments, the coupling fluid can have a freezing point and/or a phase transition point higher than the temperature of the cold plate 116. Thus, in some embodiments, the temperature of the cold plate 116, being lower than the freezing point and/or the phase transition point of the coupling fluid, can cause the formation of ice crystals in the coupling fluid which can propagate through the lesion and into the underlying tissue.

At block 1314, treatment is applied to the area to be treated, and specifically to the lesion via static application of the cold plate 116 to each of the treatment areas. In some embodiments, applying the treatment can include selecting one of the treatment areas, and holding the cold plate 116 against a surface of the skin in that treatment area for a predetermined period of time. In some embodiments, this predetermined period of time can be between approximately 5 seconds and approximately 50 seconds, between approximately 10 seconds and approximately 35 seconds, between approximately 11 seconds and approximately 30 seconds, between approximately 11 seconds and approximately 26 seconds, or any other or intermediate amount of time.

In some embodiments, and during this treatment, the temperature of the cold plate 116 can be controlled to achieve and/or maintain one or several desired temperatures. In some embodiments, this can include achieving and/or maintaining a temperature below approximately −5° C., below approximately −10° C., below approximately −15° C., between −10° C. and −25° C., to be approximately −17° C., or any other or intermediate temperature.

In some embodiments, the temperature of the cold plate 116 can be controlled according to one or several temperature profiles. In some embodiments, the temperature profile can include a first period which can be a cooling period and a second period which can be a rewarming period. The cooling period can last, in some embodiments, between 11 and 30 seconds, and the rewarming period can last, in some embodiments, between approximately 5 seconds and approximately 15 seconds, and in some embodiments, can last approximately 6 seconds. In some embodiments, and during the first period, the temperature of the cold plate can be controlled to cause the skin at the treatment location to freeze, and during the second period, the temperature of the cold plate can be controlled to cause the skin at the treatment location to be rewarmed.

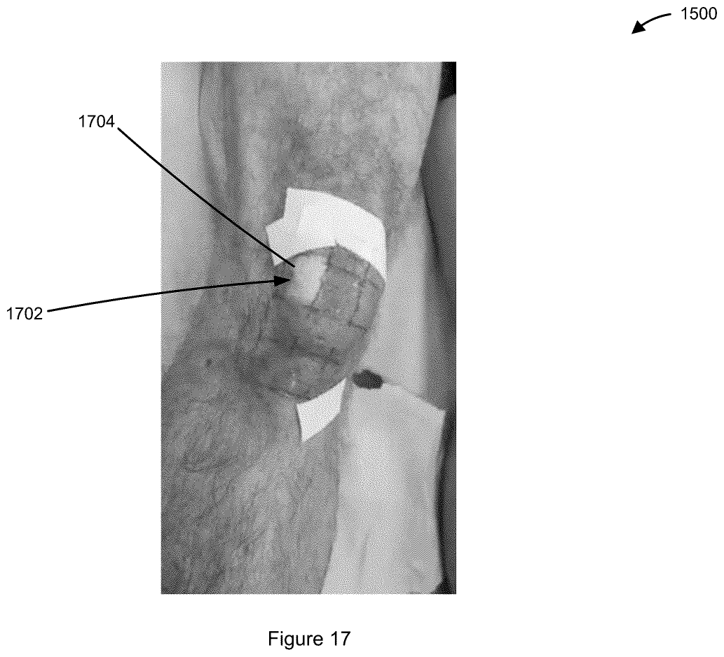

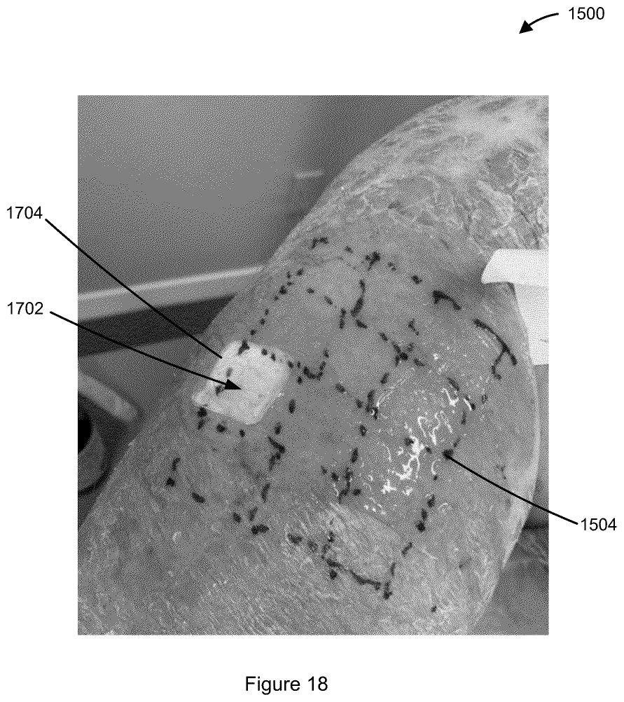

Upon the passing of the time corresponding to the delivery of the treatment, the cold plate 116 can be removed from contact with the skin in the selected one of the treatment areas. FIGS. 17 and 18 depict images of patient knees after application of a treatment to the selected one of the treatment areas. As seen, the treatment of the selected one of the treatment areas 1702 can cause treated tissue 1704, which can be frozen tissue to exhibit a change in appearance. This change in appearance can include a change in color, such as treated tissue becoming white or whiter, or becoming puffy and elevated. In some embodiments, and using the portion of the grid defining the selected one of the treatment areas 1702, it can be determined if the entirety of the selected one of the treatment areas 1702 was treated. This determination can be made by determining whether the treated tissue 1704 fills the entirety of the selected one of the treatment areas 1702. For example, as seen in FIG. 18, the treated tissue 1704 is offset with respect to the selected one of the treatment areas 1702, and does not fill the entirety of the selected one of the treatment areas 1702.

If it is determined that the entirety of the selected one of the treatment areas 1702 was not treated, then one or several remedial actions can be taken, including re-treating all or portions of the selected one of the treatment areas 1702. Alternatively, if it is determined that the entirety, or a sufficient portion of the entirety of the selected one of the treatment areas 1702 was treated, then a next treatment area 1702 can be selected and treated. This can be repeated until all of the treatment areas 1702 have been treated.

In some embodiments, a single treatment can be delivered to the lesion, and in some embodiments, multiple treatments can be delivered to the lesion over a period of time. In some embodiments, for example, a plurality of treatments can be delivered to a localized lesion over a number of months. This can include, for example, treating the localized lesion once a week, every other week, once a month, or at any other or intermediate interval. In some embodiments, treatments can be delivered for one month, for two months, for three months, for four months, for five months, for six months, between 1 and 12 months, or for any other or intermediate length of time. In some embodiments, a treatment can be delivered once a month for up to five months or until the treatment is successful.

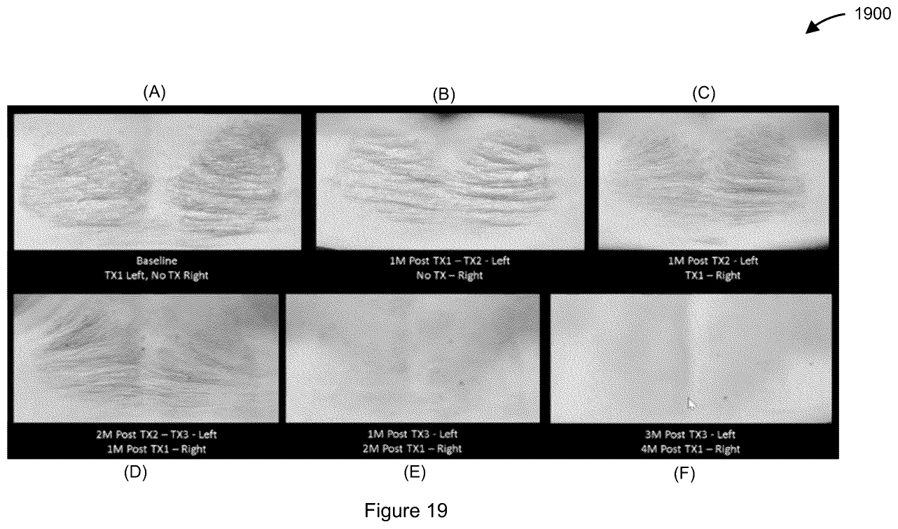

With reference now to FIG. 19, an embodiment of success from providing of treatments to a patient is shown. Specifically, FIG. 19 includes a plurality of pictures 1900 taken of two lesions on a patient over a period of time and after treatments. The top left photo, photo (A), provides a baseline for the patient, wherein one treatment has been provided to the left lesion and no treatments have been provided to the right lesion. Photo (B) depicts the lesions 1 month after the first treatments has been provided to the left lesion, and no treatments have been provided to the right lesion. Photo (C) depicts the lesion 1 month after providing the second treatment to the left lesion and at the time of providing a first treatment to the right lesion. Photo (D) depicts the lesion 2 months after providing the second treatment to the left lesion and at the time of providing the first treatment to the left lesion, and 1 month after providing the first treatment to the right lesion. Photo € depicts the lesion 1 month after providing the third treatment to the left lesion and 2 months after providing the first treatment to the right lesion. Photo (F) depicts the lesion 3 months after providing the third treatment to the left lesion and 4 months after providing the first treatment to the right lesion. As seen in FIG. 19, the treatments successfully treated the psoriatic plaques, with the plaques disappearing by photo (F).

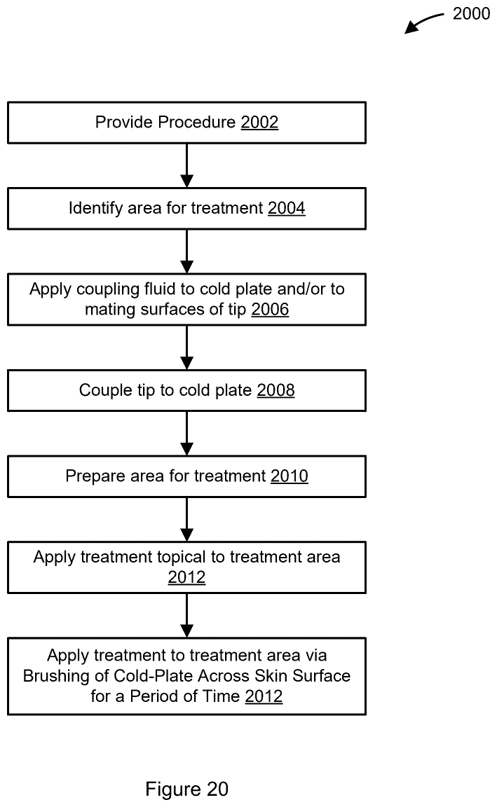

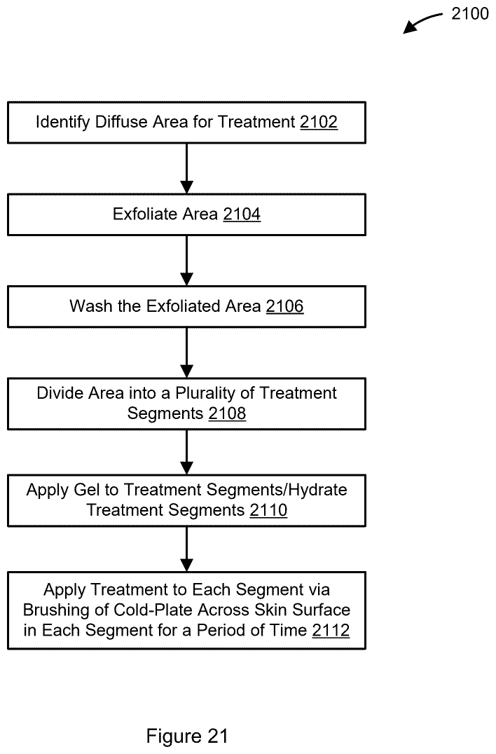

With reference now to FIGS. 20 and 21, flowcharts illustrating embodiment of processes 2000, 2100 for performing a motion cooling treatment are shown. The process 2000 can be performed utilizing a system such as a cooling treatment system 100. In some embodiment, the process 2000 can be performed to treat an area, which area can, in some embodiments, include one or several diffuse lesions. In some embodiments, the process 2000 can be performed to treat diffuse psoriatic lesions, rosacea, acne, atopic dermatitis, eczema, and/or other inflammation-driven skin ailments. In some embodiments, the motion cooling treatment can mitigate any hyperpigmentation that may arise due to inflammation, such as, for example, post inflammatory hyperpigmentation. This post inflammatory hyperpigmentation can be, in some embodiments, prevented by dissipating the inflammation that could subsequently give rise to the hyperpigmentation.

In some embodiments, the process 2000, and specifically the motion cooling treatment can be performed before, during, and/or after a medical procedure including, for example, before, during, and/or after a non-invasive or minimally invasive medical procedure, and/or before and/or after a surgical procedure. In some embodiments, for example, a non-invasive or minimally invasive medical procedure can include, for example, a non-ablative, partially-ablative, or ablative laser procedure including for example a Fraxel® laser procedure, a microneedling procedure, an RF microneedling procedure, a radiofrequency procedure, a focused ultrasound treatment, an intense pulsed light or broadband light procedure, a powered facial procedure, an injection such as a botulinum toxin injection or filler injection, or the like. In some embodiments, the motion cooling treatment can be provided withing approximately 15 minutes of the start of a non-invasive or minimally invasive medical procedure, during the non-invasive or minimally invasive medical procedure, and/or within one hour of completion of a laser procedure or other non-invasive or minimally invasive procedure. In some embodiments, the motion cooling treatment can be provided in connection with the non-invasive or minimally invasive medical procedure to thereby minimize any heat injury associated with the procedure and/or to minimize side effects arising from the non-invasive or minimally invasive medical procedure including, for example, side effects including post inflammatory hyperpigmentation. This post inflammatory hyperpigmentation can be, in some embodiments, prevented by dissipating the inflammation that could subsequently give rise to the hyperpigmentation.

In some embodiments, and in connection with a non-invasive or minimally invasive medical procedure, the motion cooling treatment can be performed using topicals compatible with open pores and/or minor incisions created through the non-invasive or minimally invasive procedure.

In some embodiments, the motion cooling treatment can be performed in connection with a surgical procedure. The surgical procedure can create an incision in a patient. The surgical procedure can include, for example, a plastic surgery such as, for example, facelift, a blepharoplasty, rhinoplasty, lip augmentation, otoplasty, genioplasty, cheek augmentation, jaw reduction, or the like. In such embodiments, the motion cooling treatment can be performed after sufficient time has passed such that the incision created by the surgical procedure is closed and is sufficiently closed to not reopen during the motion cooling treatment. In some embodiments, for example, the motion cooling treatment can be provided approximately one week after the surgical procedure. In such embodiments, the motion cooling can reduce pain, swelling, redness, edema, and/or inflammation. In some embodiments, the motion cooling treatment can further reduce scar formation including hypertrophic scar formation and/or keloid scar formation. In some embodiments, the motion cooling treatment can further decrease risk of pigment alterations arising due to the surgical procedure and specifically arising due to inflammation from the surgical procedure.

In some embodiments, the motion cooling treatment can be applied to otherwise healthy skin to suppress inflammation. In some embodiments, for example, this inflammation can arise in otherwise healthy skin due to sun exposure and the resulting sun damage. In some embodiments, this sun damage can be acute such as arising from a sun burn, and in some embodiments, this sun damage can be cumulative damage arising from extended sun exposure, such as, for example, years of sun exposure. In some embodiments, application of the motion cooling treatment to such otherwise healthy skin can reduce inflammation in that skin, which inflammation can arise from sun damage, thereby reducing premature aging of that skin. This can include decreasing the frequency of hyperpigmentation associated with aging, such as with the development of aging spots. This suppression of inflammation can further decrease development of wrinkling, skin thinning, or the like.

In some embodiments, the motion cooling treatment can be provided as part of cosmetic procedure to achieve a cosmetic effect. For example, the motion cooling treatment can be provided after a procedure such as a cosmetic or medical procedure. In some embodiments, the motion cooling treatment can, for example, mitigate swelling, inflammation, skin aging and/or indicia of skin aging, or the like. In such embodiments, the providing of the motion cooling treatment can improve aesthetic outcomes for the recipient of the treatment. In some embodiments, the aesthetic benefits of the motion cooling treatment can be permanent, however, in many instances, the aesthetic benefits of the motion cooling treatment last for some duration of time.

In some embodiments, the process 2000 can be repeated multiple times in treating an area. In some embodiments, for example, the process 2000 can be performed once, twice, three times, four times, five times, six times, seven times, eight times, nine times, ten times, fifteen times, twenty times, or any other or intermediate number of times to treat the area.

The process 2000 can begin at optional block 2002, wherein a procedure is provided. The step of block 2002 is only part of embodiments in which the motion cooling treatment is provided in connection with another procedure. Thus, in some embodiments, the step of block 2002 can be omitted.

In some embodiments, the procedure can include, for example, a medical procedure. In some embodiments, the motion cooling treatment can be provided before, during, and/or after the medical procedure. This medical procedure can be a non-invasive or minimally invasive medical procedure, and/or a surgical procedure.

The non-invasive or minimally invasive medical procedure can include, for example, a non-ablative, partially-ablative, or ablative laser procedure including for example a Fraxel® laser procedure, a microneedling procedure, an RF microneedling procedure, a radiofrequency procedure, a focused ultrasound treatment, an intense pulsed light or broadband light procedure, a powered facial procedure, an injection such as a botulinum toxin injection or filler injection, or the like. In some embodiments, the motion cooling treatment can be provided within approximately 15 minutes of the start of a non-invasive or minimally invasive medical procedure, during the non-invasive or minimally invasive medical procedure, and/or within one hour of completion of a laser procedure or other non-invasive or minimally invasive procedure. In some embodiments, the motion cooling treatment can be provided in connection with the non-invasive or minimally invasive medical procedure to thereby minimize any heat injury associated with the procedure and/or to minimize side effects arising from the non-invasive or minimally invasive medical procedure.

At block 2004, wherein an area for treatment is identified. In some embodiments, this area can comprise the area to which the procedure in block 2002 was provided, an area in which a desired aesthetic effect is desired, and/or an area containing one or several diffuse lesions such as, for example, diffuse psoriatic lesions.

In embodiments in which the motion cooling treatment is being delivered subsequent to a medical procedure, including either a surgical procedure or a non-invasive or minimally invasive medical procedure, the area for treatment can include the area to which the medical procedure was provided and/or an area around the area to which the medical procedure was provided. In some embodiments, the area around the area to which the medical procedure was provided can include areas around the area to which the medical procedure was provided including, for example, areas that are inflamed and/or that could become inflamed as a result of the procedure.

After the area for treatment has been identified, the process 2000 proceeds to block 2006, wherein a coupling fluid is applied to the cold plate 116 and/or in the receptacle 902, 1202 of the tip 119 for use in providing the motion cooling treatment. In some embodiments, this can include applying the coupling fluid to all or portions of the mating surfaces 904, 1204 of the tip 119. As discussed above, the coupling fluid can comprise a water-based fluid including: a thickener; a freeze-point depressant; and/or an antimicrobial component. to mating surfaces.

In some embodiments, the coupling fluid can comprise between approximately 90% and approximately 99% water by weight, and/or can comprise approximately 98% water by weight. In some embodiments, the coupling fluid can comprise between approximately 10% and approximately 1% thickening agent by weight, and/or can comprise approximately 2% thickening agent by weight.

In some embodiments, the coupling fluid can comprise water, a substance to either increase or decrease the freezing point of the coupling fluid such as, for example, propylene glycol, a thickening agent such as, for example, sodium carboxymethylcellulose, and/or a preservative. In some embodiments, for example, the coupling fluid can comprise between approximately 30% and approximately 90% water by weight, between approximately 50% and approximately 70% water by weight, approximately 60% water by weight, or any other or intermediate percent. In some embodiments, the coupling fluid can comprise between approximately 20% and approximately 60% substance, by weight, to increase or decrease the freezing point of the coupling fluid, approximately 40% substance, by weight, to increase or decrease the freezing point of the coupling fluid, or any other or intermediate percent of substance to increase or decrease the freezing point of the coupling fluid. In some embodiments, the coupling fluid can comprise between approximately 1% and approximately 3% thickening agent by weight, approximately 2% thickening agent by weight, or any other or intermediate percent of thickening agent.

The coupling fluid can be configured to have a viscosity sufficient to allow the coupling fluid to pass through the axial grooves 516 while coupling the tip 119 to the cold plate 116, while also being retained on and between the tip 119 and the cold plate 116. In some embodiments, this can be a viscosity of between, for example, approximately 600 centipoise and approximately 1000 centipoise. In some embodiments, the coupling fluid can have a pH or between approximately 5.0 and approximately 7.5.

In some embodiments, the coupling fluid can have a freezing point and/or a phase transition point lower than the temperature of the cold plate 116. Thus, in some such embodiments, freezing point and/or the phase transition point of the coupling fluid being lower than the temperature of the cold plate 116 prevents the formation of ice crystals in the coupling fluid.

At block 2008, the tip 119, which tip can be a smooth time 120 or a textured tip 122 is coupled to the cold plate 116. In some embodiments, and during this coupling, coupling fluid applied to the tip 119 and/or the receptacle 902, 1202 and/or air passes through one or more of the axial grooves 516 to allow the coupling of the tip 119 to the cold plate 116.

At block 2010, the area is prepared for treatment. In some embodiments, this preparation can vary based on whether a procedure was provided in block 2002 and, in the event that a procedure was provided in block 2002, the preparation can vary based on which procedure was provided in block 2002. This preparation can include, for example, washing the area, disinfecting the area, exfoliating the area, or the like. In some embodiments, one or several topicals can be used as part of this preparation.

At block 2012, a treatment topical is applied to the treatment area. In some embodiments, the treatment topical can comprise the same coupling fluid applied to the tip 119 and/or to the cold plate 116 in block 2006, and in some embodiments, the treatment topical can comprise a different fluid than the coupling fluid applied to the tip 119 and/or to the cold plate 116 in block 2006. In some embodiments, the treatment topical can be selected based on the desired effect of the motion cooling treatment and/or based on whether a procedure was provided in block 2002, and, in the event that a procedure was provided in block 2002, the treatment topical can be selected based on which procedure was provided in block 2002. In some embodiments in which the procedure abrades, perforates, or opens the skin, a treatment topical can be selected which can protect the vulnerable skin. In some embodiments, this treatment topical can serve as a lubricant to facilitate providing the motion cooling treatment and specifically that can facilitate in moving the tip 119 across the skin of the area being treated. In some embodiments, the treatment topical can further serve to improve thermal conduction and/or heat transfer between the skin being treated and the tip 119.

At block 2014, treatment is applied to the diffuse area via rubbing the cold plate 116 and/or the tip 119 across the surface of all or portions of the treatment area. The treatment can be provided for a desired duration of time. This tip 119 can, in some embodiments, be the smooth tip 120 which can be coupled to the cold plate 116. In some embodiments, and after providing the treatment, the tip 119 can be removed from the cold plate 116 and the cold plate 116 and/or the tip 119 can be washed to remove the coupling fluid applied in block 2006. In some embodiments, the tip 119 can be discarded and the cold plate 116 can be washed and then subsequently disinfected.

In some embodiments, the applying of the treatment can be monitored to ensure that the treatment is consistently and/or evenly applied. In some embodiments, this can include monitoring the location and/or movement of the application of cooling to determine that cooling is evenly applied to the skin. In some embodiments in which the cold plate is used, this can include monitoring with, for example, the motion detector 115, the position and/or movement of the cold plate. In some embodiments, if it is determined that the treatment is not being evenly applied and/or that the speed of movement of the application of cooling is significantly changing, then one or several alerts can be provided to the user, which alerts can include information directing the user to more evenly apply the treatment. This can include, for example, directing the user to increase speed of movement of the application of cooling or to decrease speed of movement of the application of cooling.

In some embodiments, the treatment can be applied to the treatment area for a predetermined period of time, which predetermined period of time can be between approximately 2 minutes and approximately 25 minutes, for between approximately 5 minutes and approximately 20 minutes, for approximately 10 minutes, or for any other or intermediate amount of time. In some embodiments, and during this period of time, treatment topical can be reapplied to the treatment are if there is insufficient treatment topical to allow the smooth rubbing of the cold plate 116 and/or tip 119 across the surface of the skin in the treatment area.

In some embodiments, and during the motion cooling treatment, the cold plate 116 and/or tip 119 attached to the cold plate 116 can be cooled to a temperature of between approximately −16° C. and 4° C., and/or between approximately −6° C. and approximately 1° C. In some embodiments, the rubbing of the cold plate 116 and/or of the tip 119 attached to the cold plate 116 across the surface of the skin of a treatment segment can cool the skin in a treatment segment to a temperature of between approximately 0° C. and 10° C., between approximately 1° C. and 6° C., between approximately 2° C. and 4° C., approximately 2° C., or any other or intermediate temperature while the cooling motion treatment is applied to that treatment segment.

In some embodiments, a single treatment can be delivered to the treatment area, and in some embodiments, multiple treatments can be delivered to the treatment area over a period of time. In some embodiments, for example, a plurality of treatments can be delivered to a treatment area over a number of months. This can include, for example, treating the treatment area once a week, every other week, once a month, or at any other or intermediate interval. In some embodiments, treatments can be delivered for one month, for two months, for three months, for four months, for five months, for six months, between 1 and 12 months, or for any other or intermediate length of time. In some embodiments, a treatment can be delivered once a month for up to five months or until the treatment is successful.

With reference now to FIG. 21, a flowchart illustrating one embodiment of a process 2100 for treating a diffuse area is shown. The process 2100 can be performed utilizing a system such as a cooling treatment system 100. In some embodiment, the process 2100 can be performed to treat a large region which can include one or several diffuse lesions. In some embodiments, the process 2100 can be performed to treat diffuse psoriatic lesions, rosacea, acne, atopic dermatitis, eczema, and/or other inflammation-driven skin ailments. In some embodiments, the motion cooling treatment can mitigate any hyperpigmentation that may arise due to inflammation, such as, for example, post inflammatory hyperpigmentation. This post inflammatory hyperpigmentation can be, in some embodiments, prevented by dissipating the inflammation that could subsequently give rise to the hyperpigmentation.

In some embodiments, the process 2100, and specifically the motion cooling treatment can be performed before, during, and/or after a medical procedure including, for example, before, during, and/or after a non-invasive or minimally invasive medical procedure, and/or before and/or after a surgical procedure. In some embodiments, for example, a non-invasive or minimally invasive medical procedure can include, for example, a non-ablative, partially-ablative, or ablative laser procedure including for example a Fraxel® laser procedure, a microneedling procedure, an RF microneedling procedure, a radiofrequency procedure, a focused ultrasound treatment, an intense pulsed light or broadband light procedure, a powered facial procedure, an injection such as a botulinum toxin injection or filler injection, or the like. In some embodiments, the motion cooling treatment can be provided withing approximately 15 minutes of the start of a non-invasive or minimally invasive medical procedure, during the non-invasive or minimally invasive medical procedure, and/or within one hour of completion of a laser procedure or other non-invasive or minimally invasive procedure. In some embodiments, the motion cooling treatment can be provided in connection with the non-invasive or minimally invasive medical procedure to thereby minimize any heat injury associated with the procedure and/or to minimize side effects arising from the non-invasive or minimally invasive medical procedure including, for example, side effects including post inflammatory hyperpigmentation. This post inflammatory hyperpigmentation can be, in some embodiments, prevented by dissipating the inflammation that could subsequently give rise to the hyperpigmentation.

In some embodiments, and in connection with a non-invasive or minimally invasive medical procedure, the motion cooling treatment can be performed using topicals compatible with open pores and/or minor incisions created through the non-invasive or minimally invasive procedure.

In some embodiments, the motion cooling treatment can be performed in connection with a surgical procedure. The surgical procedure can create an incision in a patient. The surgical procedure can include, for example, a plastic surgery such as, for example, facelift, a blepharoplasty, rhinoplasty, lip augmentation, otoplasty, genioplasty, cheek augmentation, jaw reduction, or the like. In such embodiments, the motion cooling treatment can be performed after sufficient time has passed such that the incision created by the surgical procedure is closed and is sufficiently closed to not reopen during the motion cooling treatment. In some embodiments, for example, the motion cooling treatment can be provided approximately one week after the surgical procedure. In such embodiments, the motion cooling can reduce pain, swelling, redness, edema, and/or inflammation. In some embodiments, the motion cooling treatment can further reduce scar formation including hypertrophic scar formation and/or keloid scar formation. In some embodiments, the motion cooling treatment can further decrease risk of pigment alterations arising due to the surgical procedure and specifically arising due to inflammation from the surgical procedure.

In some embodiments, the motion cooling treatment can be applied to otherwise healthy skin to suppress inflammation. In some embodiments, for example, this inflammation can arise in otherwise healthy skin due to sun exposure and the resulting sun damage. In some embodiments, this sun damage can be acute such as arising from a sun burn, and in some embodiments, this sun damage can be cumulative damage arising from extended sun exposure, such as, for example, years of sun exposure. In some embodiments, application of the motion cooling treatment to such otherwise healthy skin can reduce inflammation in that skin, which inflammation can arise from sun damage, thereby reducing premature aging of that skin. This can include decreasing the frequency of hyperpigmentation associated with aging, such as with the development of aging spots. This suppression of inflammation can further decrease development of wrinkling, skin thinning, or the like.

In some embodiments, the process 2100 can be repeated multiple times in treating a large region. In some embodiments, for example, the process 2100 can be performed once, twice, three times, four times, five times, six times, seven times, eight times, nine times, ten times, fifteen times, twenty times, or any other or intermediate number of times to treat the large region.

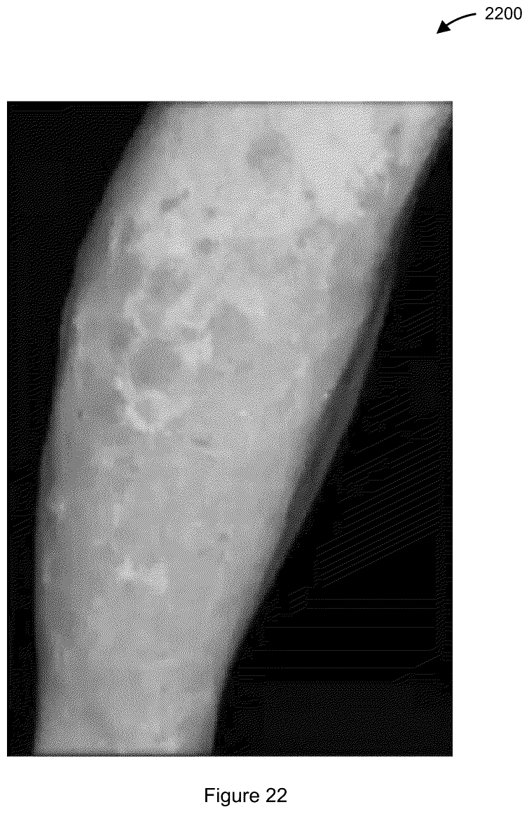

The process 2100 begins at block 2102, wherein a large area for treatment is identified. In some embodiments, this area can comprise one or several diffuse psoriatic lesions. In some embodiments, the large area can be larger than approximately 450 cm², and/or larger than approximately 400 cm². One embodiment of a diffuse lesion 2200 is shown in FIG. 22, which diffuse lesion comprises a psoriatic lesion on the leg of a patient. As seen in FIG. 22, this diffuse lesion covers a large area extending from proximate to the ankle at the bottom of the image to proximate to the knee at the top of the image.

In embodiments in which the motion cooling treatment is being delivered subsequent to a medical procedure, including either a surgical procedure or a non-invasive or minimally invasive medical procedure, the large area for treatment can be the area to which the medical procedure was provided and/or an area around the area to which the medical procedure was provided. In some embodiments, the area around the area to which the medical procedure was provided can include areas around the area to which the medical procedure was provided including, for example, areas that are inflamed and/or that could become inflamed.

After the diffuse area for treatment has been identified, the process 2100 proceeds, in some embodiments, to block 2104, wherein the area is exfoliated. While the process 2100 may, in some embodiments include exfoliation, in some embodiments, the process 2100 does not include exfoliation. In some embodiments, in which the process 2100 includes exfoliation, the exfoliation can include exfoliating some or all of the diffuse lesion in the diffuse area. As discussed above, the lesion can be exfoliated to remove any dead and/or loose tissue from the lesion to thereby facilitate thermal conductivity through the lesion and/or facilitate the propagation of ice through the lesion. In some embodiments, the lesion can be mechanically exfoliated or chemically exfoliated. In some embodiments, the exfoliation can be performed via an abrading device such as the textured tip 122, or via applying and removing an adhesive material to the plaque. For example, tape, such as bandaging tape can be applied to and removed from the plaque, thereby removing loose material from the plaque which loose material adheres to the tape.

As indicated in block 2106, upon completion of the exfoliation, the area to be treated can be washed and/or cleansed. In some embodiments, this can include washing and/or cleansing the exfoliated area to remove any debris left from the exfoliation. In some embodiments, the removal of the debris left from the exfoliation can improve the thermal conductivity and/or ice propagation through the lesion. In embodiments in which the process 2100 does not include the step of block 2104, the process 2100 can likewise omit the step of block 2106. Alternatively, and even if the step of block 2104 is omitted, the step of block 2106 can be washed and/or cleansed. In some embodiments. This washing and/or cleansing, whether performed after exfoliation or not, can be performed with water, alcohol, a cleanser, or the like. In some embodiments, water or alcohol can be used for the washing as neither leaves a residue upon drying and/or evaporating.

At block 2108, the diffuse area can be divided into a plurality of treatment segment. In some embodiments, each of these treatment segments can have a different size and/or can have approximately the same size. In some embodiments, a treatment segment can be significantly larger than the cold plate 116 and/or of a tip 119 attached to the cold plate 116. In some embodiments, each of the treatment segments can be sized such that the cold plate 116 and/or the tip 119 which can be attached to the cold plate 116 can be swept across the surface of skin in the treatment segment for the duration of the treatment. In some embodiments, each treatment segment can have a size of approximately greater than approximately the size of a hand including the palm and the fingers, and specifically has an area of less than approximately 400 cm² to approximately 450 cm². In some embodiments, each of these treatment segments can be individually treated, and treating each of the individual treatment segments leads to treating the entirety of the identified diffuse area.

At block 2110, the diffuse area, and in some embodiments, the diffuse lesion is hydrated and/or coupling fluid is applied to the diffuse area. In embodiments in which the lesion is a psoriatic plaque, the hydration of the lesion can improve the efficacy of the treatment. Hydrating the area can include applying water to the area for a period of time. This water can be applied directly to the area, and in some embodiments, a carrier containing this water can be applied to the area. In some embodiments, for example, a wet cloth or gauze can be applied to the area to thereby hydrate the area, and specifically to hydrate lesion in the area. In some embodiments, this wet cloth or gauze can be applied to the lesion for a desired period of time, which desired period of time can be, for example, between approximately 1 minute an approximately 5 minutes, between approximately 2 minutes and approximately 3 minutes, or any other or intermediate period of time. In some embodiments, the wet cloth or gauze can be applied to the lesion for a relatively longer time as a thickness of the lesion increases.

In some embodiments, the coupling fluid can hydrate the tissue of the diffuse area, facilitate cooling of the diffuse area, prevent freezing of tissue in the diffuse area, and/or decrease friction to facilitate sliding the cold plate 116 and/or tip 119 across the surface of the treatment segments.

In some embodiments, the coupling fluid can fill in air pockets and gaps in the area to be treated. Specifically, in some embodiments, the coupling fluid can fill air pockets and/or gaps in the lesion skin (e.g., resulting air pockets after the removal of debris and dead cells during the exfoliation step) and can help promote consistent and effective cooling of the plaque.

In some embodiments, the coupling fluid can be a water-based coupling fluid that can, for example, comprise water and a thickening agent. This coupling fluid can, in some embodiments, be the second coupling fluid that can be configured for use in a motion cooling treatment. In some embodiments, the coupling fluid can comprise between approximately 90% and approximately 99% water by weight, and/or can comprise approximately 98% water by weight. In some embodiments, the coupling fluid can comprise between approximately 10% and approximately 1% thickening agent by weight, and/or can comprise approximately 2% thickening agent by weight.

In some embodiments, the coupling fluid can comprise water, a substance to either increase or decrease the freezing point of the coupling fluid such as, for example, propylene glycol, a thickening agent such as, for example, sodium carboxymethylcellulose, and/or a preservative. In some embodiments, for example, the coupling fluid can comprise between approximately 30% and approximately 90% water by weight, between approximately 50% and approximately 70% water by weight, approximately 60% water by weight, or any other or intermediate percent. In some embodiments, the coupling fluid can comprise between approximately 20% and approximately 60% substance, by weight, to increase or decrease the freezing point of the coupling fluid, approximately 40% substance, by weight, to increase or decrease the freezing point of the coupling fluid, or any other or intermediate percent of substance to increase or decrease the freezing point of the coupling fluid. In some embodiments, the coupling fluid can comprise between approximately 1% and approximately 3% thickening agent by weight, approximately 2% thickening agent by weight, or any other or intermediate percent of thickening agent.

The coupling fluid can be configured to have a viscosity sufficient to allow the coupling fluid to penetrate into the plaque without running off of the plaque and/or adhere to the lesions skin. In some embodiments, this can be a viscosity of between, for example, approximately 600 centipoise and approximately 1000 centipoise. In some embodiments, the coupling fluid can have a pH or between approximately 5.0 and approximately 7.5.

In some embodiments, the coupling fluid can have a freezing point and/or a phase transition point lower than the temperature of the cold plate 116. Thus, in some such embodiments, freezing point and/or the phase transition point of the coupling fluid being lower than the temperature of the cold plate 116 prevents the formation of ice crystals in the coupling fluid, and thereby prevents the freezing the diffuse area and/or of tissue in the diffuse area.

At block 2112, treatment is applied to the diffuse area via rubbing the cold plate 116 and/or the tip 119 across the surface of each of the treatment segments for a period of time. This tip 119 can, in some embodiments, be the smooth tip 120 which can be coupled to the cold plate 116.

In some embodiments, the applying of the treatment can be monitored to ensure that the treatment is consistently and/or evenly applied. In some embodiments, this can include monitoring the location and/or movement of the application of cooling to determine that cooling is evenly applied to the skin. In some embodiments in which the cold plate is used, this can include monitoring with, for example, the motion detector 115, the position and/or movement of the cold plate. In some embodiments, if it is determined that the treatment is not being evenly applied and/or that the speed of movement of the application of cooling is significantly changing, then one or several alerts can be provided to the user, which alerts can include information directing the user to more evenly apply the treatment. This can include, for example, directing the user to increase speed of movement of the application of cooling or to decrease speed of movement of the application of cooling.

In some embodiments, applying the treatment can include selecting one of the treatment segments, and rubbing the cold plate 116 and/or the tip 119 attached to the cold plate 116 across a surface of the skin in that treatment segment for a predetermined period of time. In some embodiments, this predetermined period of time can be between approximately 2 minutes and approximately 25 minutes, for between approximately 5 minutes and approximately 20 minutes, for approximately 10 minutes, or for any other or intermediate amount of time. In some embodiments, and during this period of time, coupling fluid can be reapplied to the treatment segment if there is insufficient coupling fluid to allow the smooth rubbing of the cold plate 116 and/or tip 119 across the surface of the skin in the treatment segment.

In some embodiments, and during the motion cooling treatment, the cold plate 116 and/or tip 119 attached to the cold plate 116 can be cooled to a temperature of between approximately −16° C. and 4° C., and/or between approximately −6° C. and approximately 1° C. In some embodiments, the rubbing of the cold plate 116 and/or of the tip 119 attached to the cold plate 116 across the surface of the skin of a treatment segment can cool the skin in a treatment segment to a temperature of between approximately 0° C. and 10° C., between approximately 1° C. and 6° C., between approximately 2° C. and 4° C., approximately 2° C., or any other or intermediate temperature while the cooling motion treatment is applied to that treatment segment.

Upon the passing of the time corresponding to the delivery of the treatment to the selected treatment segment, the cold plate 116 and/or tip 119 can be removed from contact with the skin in the selected one of the treatment segments. A next, untreated treatment segment can be selected and treated. This selection and treatment of previously untreated treatment segments can be repeated until all of the treatment segments have been treated.

In some embodiments, a single treatment can be delivered to the diffuse area, and in some embodiments, multiple treatments can be delivered to the diffuse area over a period of time. In some embodiments, for example, a plurality of treatments can be delivered to a diffuse area over a number of months. This can include, for example, treating the diffuse area once a week, every other week, once a month, or at any other or intermediate interval. In some embodiments, treatments can be delivered for one month, for two months, for three months, for four months, for five months, for six months, between 1 and 12 months, or for any other or intermediate length of time. In some embodiments, a treatment can be delivered once a month for up to five months or until the treatment is successful.

This description should not be interpreted as implying any particular order or arrangement among or between various steps or elements except when the order of individual steps or arrangement of elements is explicitly described. Different arrangements of the components depicted in the drawings or described above, as well as components and steps not shown or described are possible. Similarly, some features and sub-combinations are useful and may be employed without reference to other features and sub-combinations. Embodiments of the invention have been described for illustrative and not restrictive purposes, and alternative embodiments will become apparent to readers of this patent. Accordingly, the present invention is not limited to the embodiments described above or depicted in the drawings, and various embodiments and modifications may be made without departing from the scope of the claims below.

1.-40. (canceled)

41. A method of providing a motion treatment, the method comprising:
providing a medical procedure to a procedure area of skin of a patient, wherein the medical procedure comprises a non-invasive or minimally invasive medical procedure comprising at least one of: a laser procedure; a microneedling procedure; an RF microneedling procedure; a radiofrequency procedure; a focused ultrasound treatment; an intense pulsed light or broadband light procedure; or a powered facial procedure;
identifying an area for receiving the motion treatment, the area including the procedure area;
dividing the area for receiving the motion treatment into a plurality of treatment segments;
applying a coupling fluid to the area for receiving the motion treatment; and
applying treatment to the area for receiving the motion treatment in each of the treatment segments via movingly applying cooling across a surface of tissue in each segment for a predetermined time period.

42.-50. (canceled)

51. A method of providing a motion treatment to at least a portion of skin affected by an inflammatory condition, the method comprising:
dividing the portion of skin into a plurality of treatment segments;
applying a coupling fluid to the portion of skin; and
applying treatment to the portion of skin in each of the treatment segments via movingly applying cooling across a surface of tissue in each segment for a predetermined time period.

52. The method of claim 51, wherein movingly applying cooling across the surface of tissue in each segment comprises moving a cold plate across the surface of tissue in each segment, wherein the predetermined time period is approximately 10 minutes per segment, wherein the cold plate has a temperature of between approximately −16° C. and approximately 4° C., wherein the coupling fluid has a viscosity and lubricity to facilitate moving the cold plate across the surface of the tissue.

53.-58. (canceled)

59. The method of claim 51, wherein the inflammatory condition comprises at least one of: psoriasis; atopic dermatitis; eczema; acne; or rosacea.

60. A method of providing a motion treatment, the method comprising:
providing a medical procedure to a procedure area of skin of a patient;
identifying an area for receiving the motion treatment, the area including the procedure area;
applying a treatment topical to the area for receiving the motion treatment; and
applying treatment to the area for receiving the motion treatment with a cooling treatment system, the cooling treatment system comprising:
a body comprising a chiller;
a handpiece comprising a cold plate and a cooler, wherein the cooler is thermally coupled to the cold plate and is configured to control a temperature of the cold plate; and
a cable connecting the handpiece and the body, and wherein a coolant is configured to circulate between the chiller and the cooler via the cable.

61. The method of claim 60, wherein the medical procedure comprises a non-invasive or minimally invasive medical procedure, wherein the non-invasive or minimally invasive medical procedure comprises at least one of: a laser procedure; a microneedling procedure; an RF microneedling procedure; a radiofrequency procedure; a focused ultrasound treatment; an intense pulsed light or broadband light procedure; or a powered facial procedure.

62.-63. (canceled)

64. The method of claim 60, wherein the medical procedure comprises a surgical procedure, wherein providing the surgical procedure creates an incision, and wherein the treatment is applied after the incision has closed.

65. (canceled)

66. The method of claim 60, further comprising coupling a treatment tip to the cold plate, and wherein applying treatment to the area for receiving the motion cooling treatment comprises contacting the treatment tip to the surface of tissue in the area and moving the treatment tip coupled to the cold plate across the surface of tissue in the area for a predetermined time period.

67. The method of claim 66, wherein the predetermined time period is approximately 10 minutes per segment.

68. The method of claim 66, wherein the cold plate has a temperature of between approximately −16° C. and approximately 4° C.

69. The method of claim 66, wherein the treatment tip comprises a smooth tip.

70. The method of claim 66, wherein the cold plate comprises:
a distal protrusion;
a camming surface extending proximally and radially away from a proximal end of the distal protrusion;
a retention depression connecting to a proximal end of the camming surface.

71. The method of claim 70, wherein the cold plate further comprises a plurality of axial grooves located around a perimeter of a junction of the proximal end of the camming surface and the retention depression.

72. The method of claim 71, wherein the retention depression extends to a retention depth below the proximal end of the camming surface, and wherein the axial grooves have a groove depth equal to the retention depth.

73. The method of claim 72, wherein the tip comprises:
an exterior housing defining a receptacle; and
a retention feature located inside the receptacle, wherein the receptacle configured to receive at least portions of the cold plate, and wherein the retention feature is configured to engage with the retention depression of the cold plate to couple the tip to the cold plate.

74. The method of claim 73, further comprising:
applying coupling fluid to at least one of the cold plate and the receptacle of the tip; and
coupling the tip to the cold plate.

75. The method of claim 74, wherein coupling the tip to the cold plate expels at least a portion of the coupling fluid via at least one of the plurality of axial grooves.

76. The method of claim 74, wherein the coupling fluid is different than the treatment topical, wherein the coupling fluid has a freezing point lower than a temperature of the cold plate.

77. (canceled)

78. The method of claim 60, wherein the cooler comprises a thermoelectric cooler (TEC).

79. The method of claim 78, wherein applying the treatment to the area for receiving the motion treatment with a cooling treatment system comprises controlling a temperature of the cold plate with the cooler.
